(19) United States
(12) Patent Application Publication
(10) Pub. No.: US 2023/0016357 A1
(43) Pub. Date: Jan. 19, 2023

(54) DETERMINISTIC HYBRIDOMA GENERATION VIA MICROFLUIDICS

(71) Applicant: SCRIBE BIOSCIENCES, INC., San Francisco, CA (US)

(72) Inventors: Russell Cole, San Francisco, CA (US); Maithreyan Srinivasan, San Francisco, CA (US)

(21) Appl. No.: 17/690,427

(22) Filed: Mar. 9, 2022

Related U.S. Application Data
(60) Provisional application No. 63/159,151, filed on Mar. 10, 2021.

(51) Int. Cl. C12M 1/42; C12N 15/02; C12N 5/16

(52) U.S. Cl. CPC C12M 35/02; C12N 15/02; C12N 5/163

(57) ABSTRACT

The present invention provides compositions, systems, kits, and methods for combining a. single myeloma cell and a single B-cell (e.g., from an animal exposed to a desired antigen) via discrete entity (e.g., droplet) microfluidics. In certain embodiments, a microfluidic device is used to merge a discrete entity containing a B-cell, and a discrete entity containing a myeloma cell, and a discrete entity containing gellable material, at a merger region via a trapping element in order to generate a combined discrete entity. In further embodiments, the combined discrete entity is treated such that a gelled discrete entity is formed.

The present application claims priority to U.S. Provisional application Ser. No. 63/159,181, filed Mar. 10, 2021, which is herein incorporated by reference in its entirety.

### FIELD OF THE INVENTION

The present invention provides compositions, systems, kits, and methods for combining a single myeloma cell and a single B-cell (e.g., from an animal exposed to a desired antigen) via discrete entity (e.g., droplet) microfluidics. In certain embodiments, a microfluidic device is used to merge a discrete entity containing a B-cell, and a discrete entity containing a myeloma cell, and a discrete entity containing gellable material, at a merger region via a trapping element in order to generate a combined discrete entity. In further embodiments, the combined discrete entity is treated such that a gelled discrete entity is formed. In other embodiments, the gelled discrete entity is treated such that the myeloma cell and B-cell fuse, generating a hybridoma, which is assayed to determine if a desired monoclonal antibody is secreted therefrom.

### BACKGROUND

Monoclonal antibodies (mAb) target specific antigens (unique antigenic determinants or epitopes) and are produced by B-cells. Each B-cell secretes a unique antibody that targets a unique epitope. However, B-cells are short-lived and therefore are difficult to maintain in culture for extended periods of time making them ill-suited for obtaining pure mAbs in large amounts. To extend their lifetime, B-cells isolated from an immunized animal are fused with tumor cells to form hybrids or hybridomas, where B-cells acquire the proliferative properties of tumor cells, propagate in culture indefinitely and serves as a source of purified mAbs. MAb therapeutics were a $115 billion business in 2018 and is projected to increase to $300 billion by 2025. Seventy-nine mAb therapies have been approved by the FDA and at least 570 mAb candidates are in clinical trials[1].

The process of generating a hybridomas is very inefficient. Several factors contribute to this inefficiency. The starting point for hybridoma generation is isolated splenocytes typically from mice. Splenocytes are mixed population of multiple cell types and only 5% of the splenocytes are mature B-cells. Mature B-cells are enriched using established methods and fused with myeloma cells to generate hybridomas in bulk. Bulk cell fusion is typically performed with the help of either polyethylene glycol (PEG) or electro-cell manipulators that facilitate electro-fusion. The frequency of B-cells that express antibodies for the antigen of interest can be as low as 1 in 10,000[2] and cell fusion efficiencies using conventional methods are in the order of 10⁻⁵ to 10⁻⁶. Thus, the chance of obtaining an antigen-specific hybridoma is at least 1 in a billion[3]. Therefore, there is critical need for new methods to improve the efficiency of hybridoma generation.

Hybridoma generation efficiencies have been improved to 1 in 1,000 to 1 in 10,000 by the use of Epstein-Barr virus transformed human B-cells and heterohybridoma cell lines created from murine/human cell fusions[3]. But this improvement while improving overall odds of identifying a B-cell secreting antibodies for the antigen of interest to at least 1 in 10 million, still requires ultra-high throughput technologies to sample millions of hybridomas. Microfluidic methods have been used to increase the efficiency of cell fusion randomly bringing cells into contact either by alternating current fields in flow-through devices or immobilization techniques using biotin-streptavidin coatings[3 and references therein]. To make the cell pairing more deterministic, microfluidic traps were used to pair different cell types with 50% pairing and fusion efficiencies, but throughputs of 6,000 traps does not adequately address the need to screen millions of cells[4].

Lack of deterministic pairing of single B-cells and myeloma cells and the ability to sample hundreds of thousands to millions of cells are two main issues with current technologies for not addressing market needs. After hybridomas are generated, many of the hybridomas may not survive because of chromosomal instability or not enough antibodies would be secreted. Ability to sample the same clonal B-cell multiple times is important.

### SUMMARY OF THE INVENTION

The present invention provides compositions, systems, kits, and methods for combining a single myeloma cell and a single B-cell (e.g., from an animal exposed to a desired antigen) via discrete entity (e.g., droplet) microfluidics. In certain embodiments, a microfluidic device is used to merge a discrete entity containing a B-cell, and a discrete entity containing a myeloma cell, and a discrete entity containing gellable material, at a merger region via a trapping element in order to generate a combined discrete entity. In further embodiments, the combined discrete entity is treated such that a gelled discrete entity is formed. In other embodiments, the gelled discrete entity is treated such that the myeloma cell and B-cell fuse, generating a hybridoma, which is assayed to determine if a desired monoclonal antibody is secreted therefrom.

In some embodiments, provided herein are methods comprising: a) flowing a first and second discrete entity in a carrier fluid in a microfluidic device, wherein the microfluidic device comprises: i) an inlet channel, ii) a sorting channel in fluid communication with the inlet channel, iii) first and second outlet channels in fluid communication with the sorting channel, wherein the first outlet channel comprises a merger region, iv) a sorting element positioned in proximity to the sorting channel, and v) a trapping element positioned in proximity to the merger region, and wherein the first discrete entity comprises one, and only one, myeloma cell and is free of other types of cells, wherein the myeloma cell comprises: A) a first detectable label; and B) optionally, an outer surface displaying a first moiety; wherein the second discrete entity comprises one, and only one, B-cell and is free of other types of cells, wherein the B-cell comprises: i) a second detectable label, and ii) optionally a second moiety that forms a binding pair with the first moiety; and wherein the flowing causes the first and second discrete entities to pass through the inlet channel to the sorting channel; b) selectively sorting the first and second discrete entities in the sorting channel to the first outlet channel; c) trapping the first or second discrete entity in the merger region via the trapping element such that they combine to form at least part of a first combined discrete entity.

In certain embodiments, the methods further comprise: combining, in any order, a third discrete entity with the first and second discrete entities at the merger region to form at least part of the first combined discrete entity, wherein the third discrete entity comprises gellable material and is free from cells. In additional embodiments, the methods further comprise releasing the first combined discrete entity from the merger region such that it flows into a downstream area. In other embodiments, the downstream area is a collection area, or a receptacle external to the microfluidic device.

In additional embodiments, the methods further comprise: d) treating the first combined discrete entity such that the gellable material gels, thereby forming a first gelled discrete entity. In some embodiments, the treating comprises incubating the first combined discrete entity at a temperature that causes the gellable material to gel. In other embodiments, the gellable material comprises low melt agarose.

In particular embodiments, the methods further comprise: combining, in any order, a fourth discrete entity with the first, second, and third discrete entities at the merger region to form at least part of the first combined discrete entity, wherein the fourth discrete entity comprises a gel activator agent. In additional embodiments, the gel activator agent comprises an agent configured to release Ca²⁺ ions.

In further embodiments, the methods further comprise: releasing the first combined discrete entity from the merger region such that it flows into a downstream area. In other embodiments, the methods further comprise: incubating the first combined discrete entity such that a first gelled discrete entity is formed. In some embodiments, the gellable material comprises low melt alginate. In certain embodiments, the incubating is conducted for a time sufficient for the gel activator agent to convert all, or nearly all, of the gellable material into a gel.

In some embodiments, the first combined discrete entity further comprises gellable material. In additional embodiments, the methods further comprise comprising releasing the first combined discrete entity from the merger region such that it flows into a downstream area. In other embodiments, the methods further comprise: treating the first combined discrete entity such that the gellable material gels, thereby forming a first gelled discrete entity. In additional embodiments, the treating comprises incubating for a sufficient time and/or incubating at a particular temperature.

In other embodiments, the methods further comprise: breaking the first gelled discrete entity. In additional embodiments, the breaking comprises treating the first gelled discrete entity with agarase. In particular embodiments, the breaking comprises treating the first gelled discrete entity with an alginate lyase. In other embodiments, the breaking comprises treating the first gelled discrete entity with a calcium ion sequestering agent and/or suspending the first gelled discrete entity in a buffer comprising phosphate or citrate.

In additional embodiments, the methods further comprise: treating the first gelled discrete entity, either broken or not broken, such that the B-cell and myeloma cell fuse to generate a first hybridoma. In further embodiments, the treating comprises treatment with electrofusion methods. In some embodiments, the treating comprises contacting the first gelled discrete entity with a cell fusion reagent. In other embodiments, the cell fusion reagent comprises polyethylene glycol. In additional embodiments, the methods further comprise: propagating hybridomas (in gelled droplets) (e.g., propagate for a few cell divisions), subjecting the hybridoma to an antigen binding assay and/or functional assay and sorting hybridomas (in gelled droplets) for their functional signatures.

In some embodiments, provided herein are methods using the microfluidic devices described herein, comprising: A) flowing: i) a discrete entity containing said hybridoma (hybridoma discrete entity) in a carrier fluid, ii) a discrete entity containing detection reagents (detection discrete entity) in said carrier fluid; and iii) a discrete entity containing gellable material (gellable discrete entity); B) selectively sorting said hybridoma, detection, and gellable discrete entities in a sorting channel to a first outlet channel; and C) trapping said hybridoma, detection, and gellable discrete entities in a merger region via a trapping element such that they combine forming a second combined discrete entity. In further embodiments, the methods further comprise: releasing the second combined discrete entity from the merger region such that it flows into a downstream area. In other embodiments, the methods further comprise: incubating and/or treating said second combined discrete entity such that: 1) said detection reagents generate a detectable signal if said hybridoma secretes a desired monoclonal antibody; and 2) said gellable material gels, thereby generating a second gelled discrete entity. In other embodiments, the detection reagents comprise: i) binding assay reagent, or ii) functional assay reagents.

In some embodiments, the methods further comprise, in the microfluidic devices described herein, D) flowing: i) said second gelled discrete entity in a carrier fluid, ii) a discrete entity containing sequencing reagents (sequencing discrete entity) in said carrier fluid; and iii) a discrete entity containing lysis reagent (lysis discrete entity); E) selectively sorting said second gelled discrete entity into a first outlet channel based on detecting said detectable signal; F) selectively sorting said sequencing and lysis discrete entities into said first outlet channel; and G) trapping said second gelled entity, sequencing, and lysis discrete entities in a merger region via a trapping element such that they combine forming a third combined discrete entity. In particular embodiments, the methods further comprise: releasing said third combined discrete entity from said merger region such that it flows into a downstream area. In other embodiments, the methods further comprise: treating said third combined discrete entity such that: i) said hybridoma is lysed; ii) nucleic acids from said hybridoma are reverse transcribed, iii) said third combined discrete entity is broken, and iv) the reverse transcribed nucleic acids are amplified generating amplified nucleic acid.

In certain embodiments, the first detectable label comprises a first fluorescent dye, and the second detectable label comprises a second fluorescent dye different from the first fluorescent dye. In other embodiments, the first and second moieties are present. In some embodiments, the first and second moieties are selected from the group consisting of: oligonucleotides, aptamers, protein, receptors, antibody, and binding region of an antibody.

In some embodiments, provided herein are methods comprising: a) flowing a first, second, and third discrete entities in a carrier fluid in a microfluidic device, wherein said microfluidic device comprises: i) an inlet channel, ii) a sorting channel in fluid communication with said inlet channel, iii) first and second outlet channels in fluid communication with said sorting channel, wherein said first outlet channel comprises a merger region, iv) a sorting element positioned in proximity to the sorting channel, and v) a trapping element positioned in proximity to said merger region, and wherein said first discrete entity comprises one, and only one, hybridoma cell and is free of other types of cells, wherein said second discrete entity comprises detection reagents, and wherein said third discrete entity comprises gellable material; b) selectively sorting said first, second, and third discrete entities in a sorting channel to a first outlet channel; and c) trapping said first, second, and third discrete entities in a merger region via a trapping element such that they combine forming a first combined discrete entity.

In certain embodiments, the methods further comprise: d) releasing said first combined discrete entity from said merger region such that it flows into a downstream area. In additional embodiments, the methods further comprise: e) incubating and/or treating said first combined discrete entity such that: 1) said detection reagents generate a detectable signal if said hybridoma secretes a desired monoclonal antibody; and 2) said gellable material gels, thereby generating a first gelled discrete entity. In other embodiments, the detection reagents comprise: i) binding assay reagents, or ii) functional assay reagents. In additional embodiments, the methods further comprise, in a microfluidic device as described herein, f) flowing: i) said first gelled discrete entity in a carrier fluid, ii) a fourth discrete entity comprising sequencing reagents in said carrier fluid; and iii) a fifth discrete entity comprising a lysis reagent; g) selectively sorting said second gelled discrete entity into a first outlet channel based on detecting said detectable signal; h) selectively sorting said fourth and fifth discrete entities into said first outlet channel; and i) trapping said second gelled, fourth, and fifth discrete entities in a merger region via a trapping element such that they combine forming a second combined discrete entity.

In particular embodiments, the methods further comprise: releasing said second combined discrete entity from said merger region such that it flows into a downstream area. In other embodiments, the methods further comprise: treating said second combined discrete entity such that: i) said hybridoma is lysed; ii) nucleic acids from said hybridoma are reverse transcribed, iii) said second combined discrete entity is broken, and iv) the reverse transcribed nucleic acids are amplified generating amplified nucleic acid. In some embodiments, the methods further comprise: identifying at least part of the sequence of the monoclonal antibody (e.g., at least the variable region) secreted by said hybridoma.

In additional embodiments, steps a)-c) are performed: A) in 2 milliseconds (mS) or less; B) is about 1 mS; C) in about 0.5-1.0 mS, D) in about 10-100 mS; or E) about 100-1000 mS. In further embodiments, the methods further comprise: repeating steps a)-c) at least 100 times such that a total of at least 100 combined discrete entities are formed. In other embodiments, the methods further comprise: repeating steps a)-c) at least 1000 times such that a total of at least 1000 combined discrete entities are formed. In other embodiments, the methods further comprise: repeating steps a)-c) at least 1000 times such that a total of at least 10000 combined discrete entities are formed. In additional embodiments, the repeating steps a)-c) at least 100,000 times such that a total of at least 100,000 combined discrete entities are formed. In other embodiments, the 100 or the 1000 discrete entities are formed: A) in 2 seconds or less; B) is about 1 second; C) in about 30-60 seconds. In other embodiments, the 10,000 or the 100,000 discrete entities are formed: A) in 20 seconds or less; B) is about 10 seconds; C) in about 300-600 seconds.

In additional embodiments, the myeloma cell and/or the B-cell are mammalian cells or human cells. In further embodiments, the methods further comprise: releasing the first combined discrete entity from the discrete entity merger region by deactivating, decreasing, or reversing the trapping element such that first combined discrete entity flows out of the first outlet channel. In other embodiments, the sorting element comprises a first sorting electrode that exert an electromagnetic force sufficient to sort a discrete entity in the sorting channel to the first outlet channel. In some embodiments, the electromagnetic force is a dielectrophoretic force or an electrophoretic force. In further embodiments, the microfluidic device further comprises a second and/or third sorting electrode.

In some embodiments, the trapping element exerts an electromagnetic force, exerts a mechanical force, or a combination thereof sufficient to trap the first and second discrete entities in the discrete entity merger region for a time sufficient for the discrete entities to combine to form a combined discrete entity. In certain embodiments, the trapping element comprises a first trapping electrode that exerts an electromagnetic force sufficient to trap discrete entities in the merger region for a time sufficient for discrete entities to combine to form a combined discrete entity. In additional embodiments, the electromagnetic force is a dielectrophoretic force or an electrophoretic force. In further embodiments, the first, second, third, and/or fourth discrete entities are droplets. In particular embodiments, the droplets comprise an aqueous fluid which is immiscible in the carrier fluid. In some embodiments, the carrier fluid comprises oil or water.

In particular embodiments, the discrete entities have a diameter of from about 1 μm to 1000 μm. In other embodiments, the discrete entities have a volume of from about 1 femtoliter to about 1000 nanoliters, or from 10 to 800 picoliters.

In some embodiments, provides herein are compositions, systems, and kits comprising: a) first and second discrete entities in a carrier fluid, and/or b) a combined discrete entity in a carrier fluid, wherein the combined discrete entity is a combination of the first and second discrete entities, wherein the first discrete entity comprises one, and only one, myeloma cell and is free of other types of cells, wherein the myeloma cell comprises: A) a first detectable label; and B) optionally, an outer surface displaying a first moiety; and wherein the second discrete entity comprises one, and only one, B-cell and is free of other types of cells, wherein the B-cell comprises: i) a second detectable label, and ii) optionally a second moiety that forms a binding pair with the first moiety. In certain embodiments, the systems further comprise: c) a microfluidic device comprising: i) an inlet channel, ii) a sorting channel in fluid communication with the inlet channel, iii) first and second outlet channels in fluid communication with the sorting channel, wherein the first outlet channel comprises a merger region, iv) a sorting element positioned in proximity to the sorting channel, and v) a trapping element positioned in proximity to the merger region.

In additional embodiments, the compositions, systems, and kits further comprise a plurality of the first discrete entities and/or a plurality of the second discrete entities. In other embodiments, each of the first and second discrete entities comprises a droplet, and/or wherein the combined discrete entity comprises a droplet. In other embodiments, the discrete entities have a volume of from about 1 femtoliter to about 1000 nanoliters (e.g., 1 . . . 50 . . . 300 . . . 1000), or from 10 to 800 picoliters.

In certain embodiments, the B-cells are from a rabbit, mouse, chicken, human, alpaca, camelid, or other animal. In particular embodiments, the myeloma cell is a homomyelomas cell or a heteromyelomas cell.

### DESCRIPTION OF THE FIGURES

FIG. 1A shows exemplary droplet components including a B-cell (e.g., from a mouse immunized with an antigen of interest) with a green dye and first oligo on its surface, a myeloma cell with a blue dye and second oligo on its surface to hybridize to the first oligo, and a droplet with reagents (e.g., containing a gellable agent, such as agarose or alginate).

FIG. 1B shows an exemplary flow path for discrete entities containing B-cell, myeloma cell, and gellable agent, including: a) flowing the three different discrete entities in a carrier fluid and sorting based on presence of dye or absence of dye into either waste channel or channel with a trapping and merger region (to create a combined discrete entity); b) collecting the combined discrete entities, each having one B-cell linked to one myeloma cells by surface oligo, and gellable agent; c) treating the combine discrete entities (e.g., incubate for certain amount of time or cooler temperature) to generate gelled discrete entities; d) treating the gelled discrete entities with a fusion methodology, such as PEG 1500 or electrofusion methods, such that hybridomas are generated; and e) collecting the hybridomas with some or all of the hydrogel material removed.

FIG. 2A shows exemplary droplet components, including a hybridoma (e.g., labelled with green), a detection bead, and binding and/or functional assay reagents.

FIG. 2B shows an exemplary flow path with four main steps: 1) sorting and assembling the discrete entities containing the hybridoma, detection bead, and reagents (e.g., gellable material; functional assay or binding assay reagents) to generate a first combined discrete entity; 2) incubate the first combined discrete entity formed to form gelled discrete entity and allow assay reaction to occur; 3a) sorting of the gelled discrete based on presence of desired activity; 3b) combine the first combined discrete entity with the desired hybridoma with one or more discrete entities with lysis reagent and sequencing reagents, to generate a second combined discrete entity; and 4) treat the second combined discrete entity such that hybridoma is lysed and nucleic acid sequences therein are sequences (e.g., to identify sequence of the desired monoclonal antibody).

FIG. 3 provides a block schematic diagram of an example microfluidic device having an inlet channel, a sorting channel, a sorting element, first and second outlet channels, a trapping element, a discrete entity merger region, and upstream and a downstream regions.

FIG. 4 provides an image of a microfluidic device having a spacer fluid channel, a bias fluid channel, a laminating oil inlet channel, a concentric sorter channel, a flow divider, and a recess according to embodiments of the present disclosure.

FIG. 5 provides images of a microfluidic device having a concentric sorter channel, a recess, and an approximately triangular downstream region according to embodiments of the present disclosure.

FIGS. 6A-D show a zoomed-out view of an integrated droplet sorter-combiner. A droplet with a desired fluorescent signature is detected as it enters the droplet sorting region (A), the sorting electrode is actuated to redirect the drop towards the assembly lane (B), and the sorted droplet merges with the droplet-in-assembly at the DEP trap (C). Following assembly, the DEP trap is turned off to release the droplet (D).

FIGS. 6E-H show a close-up of the merging process. 4 droplets are sorted by their fluorescent signature (pseudocolored) and directed to the DEP trap for merging (E). As the droplets encounter the actuated trap, they are sequentially merged into the assembled droplet (F-G). The electrode is then temporarily turned off so the assembled droplet may be released and recovered downstream (H).

FIG. 7 provides a schematic flow diagram of a method of selectively combining discrete entities using a microfluidic device according to embodiments of the present disclosure.

FIGS. 8A-C provides a schematic showing example configurations for trapping a discrete entity. FIG. 8A shows a bipolar electrode pair embedded in the same side wall of a channel. FIG. 8B shows a bipolar electrode pair embedded on opposite sides of channel. FIG. 8C shows bipolar electrode pair embedded in the floor or ceiling of a channel.

FIGS. 9A-E provides a schematic showing example configurations for directing discrete entities to a discrete entity merger region. FIG. 9A shows application of a lamination flow to confine the laminar flow containing the droplet to the side wall of the channel. FIG. 9B shows a partial height flow divider that allows fluid, but not droplets to enter the center portion of the channel. FIG. 9C shows a configuration where a groove of similar height to the droplet dimensions is patterned near the side wall of a channel, while the rest of the channel is constructed with a reduced height to exclude droplets. FIG. 9 D shows a porous flow divider that allows fluid, but not droplets to enter the center portion of the channel. FIG. 9E shows a partial height flow dividers that direct droplets to a trap at the center of the microfluidic channel.

FIGS. 10A-C provide a schematic showing an example embodiment wherein trapping is facilitated by a mechanical valve. FIG. 10A shows an initial stage where the discrete entities are trapped by the valve. FIG. 10B shows a second stage wherein the discrete entities have been combined, e.g. due to electrical, chemical, or other means. FIG. 10C shows a third stage where the combined discrete entity is released by opening the valve and carried downstream.

FIGS. 11A-C provide a schematic showing example embodiments with different channel geometries in proximity to an electromagnetic trapping element. FIG. 11A shows a discrete entity merger region upstream of a bend in the channel wall. FIG. 11B shows a discrete entity merger region in a lateral facet in the channel wall. FIG. 11C shows a discrete entity being trapped in a region that is vertically taller than the main channel.

FIG. 12 shows exemplary droplet workflow. In the first step, discrete entities with B-cells, antigen capture beads, and assay reagents, are selectively sorted into a trapping and merger region (to create a combined discrete entity). Next, the combined discrete entities are collected (each having one B-cell with assay reagents) and incubated for a certain amount of time such that an assay reaction can occur to identify characteristics of the B-cell (e.g., such as if it secretes the desired antibody or secretes an antibody with high enough binding capacity). Next, discrete entities with a single myeloma cell, gellable material, and the combined discrete entities are sorted and trapped at a merger region (to create a second combined discrete entity). B-cells sorted to the trapping area contain the desired antibody via the assay, while undesired B-cells are sorted to the discard channel. Next, the second combined discrete entities are collected and then treated (e.g., incubated for certain amount of time or cooler temperature) to generate gelled discrete entities. The gelled discrete entities are then treated with a fusion methodology, such as PEG 1500 or electrofusion methods, such that hybridomas are generated. Finally, the hybridomas are collected and some or all of the hydrogel material is removed.

### DETAILED DESCRIPTION

The present invention provides compositions, systems, kits, and methods for combining a single myeloma cell and a single B-cell (e.g., from an animal exposed to a desired antigen) via discrete entity (e.g., droplet) microfluidics. In certain embodiments, a microfluidic device is used to merge a discrete entity containing a B-cell, and a discrete entity containing a myeloma cell, and a discrete entity containing gellable material, at a merger region via a trapping element in order to generate a combined discrete entity. In further embodiments, the combined discrete entity is treated such that a gelled discrete entity is formed. In other embodiments, the gelled discrete entity is treated such that the myeloma cell and B-cell fuse, generating a hybridoma, which is assayed to determine if a desired monoclonal antibody is secreted therefrom.

Droplet microfluidics have been used for particular functional screens of hybridoma cells[5] but not for deterministic assemblies of B-cells and myeloma cells as a way to increase the efficiency to form hybridomas. B-cells from mice or humans and myeloma cells that were previously characterized for their ability to form hybridomas are, in certain embodiments, encapsulated as single-cells and merged using the Microenvironment on demand (MOD) device[6]-[8] (herein incorporated by reference in their entireties, and specifically for teaching the MOD devices) to create, for example, 100,000 to 1 million assembled hydrogel droplets[9] that contain a single B-cell and a single myeloma cell. Hydrogel droplets can be made using low-melt agarose or alginate. Alginate hydrogel formation occurs in the presence of Ca2+-EDTA complex and the gelation can be timed through the controlled release of Ca2+ in the presence of acetic acid[9]. To assist cell fusion, lipid-oligos (or other binding partners) with complementary sequences may be used to decorate B-cells and myeloma cells such that oligo is exposed to the extracellular space anchored to the cell membrane via the lipid moiety[10][11]. The complementary sequences are used to bring the B-cell and myeloma cell in close proximity and aid in cell fusion. Similar alternative approaches would use bispecific antibodies or engineered myeloma cells that express biotin acceptor peptide in the extracellular space that can be biotinylated in the presence of exogenously added biotin and biotin ligase[12]. Streptavidin labeled anti-CD21 can be used to decorate B-cells and cell fusion can be accelerated through biotin-streptavidin interaction. The use of biotin acceptor peptide expressing myeloma cells in forming hybridomas also has the added benefit of a direct connection between the single hybridoma cell and its secreted antibody[12].

In certain embodiments, the droplets containing B-cell/myeloma cell doublets are broken and the doublet cells are subjected to cell-fusion using establish methods[13][14][15]. Chemical fusion methods generally function at low efficiencies and use the water-exclusion properties of PEG1500 to hasten cell fusion. Chemical fusion efficiencies can potentially be improved by breaking emulsions containing hydrogel droplets (of suitable pore sizes) that contain the individual B-cell/myeloma pairs (separated from each pair due to hydrogel boundaries) and treating the collected droplets with PEG1500. Alternatively, cells in droplets can be fused using electrofusion methods where droplets containing cell pairs pass through an array of electrodes that pulse at preset strengths[15].

In certain embodiments, the initial B-cells are purified mature plasma cells (e.g., generated using kits to isolate such cells, such as sold by Miltenyi) from an animal (e.g., mouse or rat) immunized with an antigen of interest. In certain embodiments, the plasma-cells are labeled with a generic dye like Celltracker and sorted using Mod.

In certain embodiments, the hydrogels formed herein are broken prior to further manipulation. For example, agarose hydrogels may be broken by agarase (e.g., sold by NEB). In other embodiments, the hydrogel is composed of alginate. In such embodiments, alginate lyase may be employed to break such hydrogels. In other embodiments, sequestering agents for calcium ions are employed. For example, alginate can be broken down under mild conditions with little loss of activity of the cells and the activity is often stable for extended periods of time. Temperatures can be 0-100° C. and the pH neutral but any buffers used must not contain citrate or phosphate. These anions will remove calcium ions from the gel and can lead to its breakdown, although Birnbaum et al. (1981), have developed methods for stabilizing alginate gels in phosphate-containing media. The cells can be recovered if necessary be adding a sequestering agent for the calcium ions (such as polyphosphate or EDTA); once the calcium ions are removed from the gel, its structure is lost and it changes to a liquid with the cells suspended in it. In other embodiments, suspending alginate hydrogels in phosphate or citrate containing buffer to extract the calcium is used to break the hydrogel discrete entities herein.

In certain embodiments, the B-cell and myeloma cell are fused to generate a hybridoma using chemical fusion. In certain embodiments, chemical infusion involves merging a PEG droplet into the B-cell/myeloma cell droplet. In certain embodiments, the B-cell and myeloma cell comprise cell tethers that binding to each other. As such, this allows the cells to remain associated with each other, even after the hydrogel is broken.

In certain embodiments, the microfluidic device comprises a microenvironment on Demand (MOD) device, described in U.S. Provisional application Ser. No. 62/847,791, which is incorporated by reference herein. In certain embodiments, the MOD platform is composed of an combination of three technologies: 1) a deterministic single-cell droplet sorter and droplet-assembler that can selectively assemble cells and reagents 2) cell-based assays adapted to single-cell (hybridomas) in droplets (e.g., to determine if desired antibody is generated by a particular hybridoma), and 3) molecular biology methods that can capture mRNAs corresponding to hybridomas and process them for DNA sequencing.

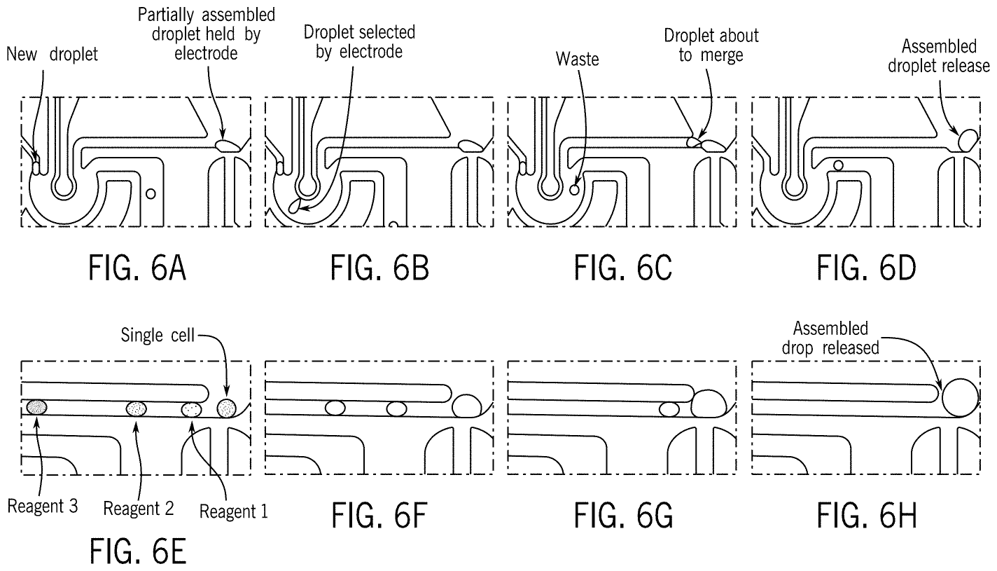

MOD performs a cyclic buildup and release of designer droplets through the merging of select droplets on a defined dielectrophoretic trapping position inside the microfluidic device (FIGS. 6A-H). This approach is advantageous because it is less prone to contamination, higher throughput, and requires fewer moving parts than other devices. The flexible nature of the MOD platform makes it a well-suited technology to perform integrated and functional cell-cell, cell-ECM interaction analysis and link any perturbations to select expressed gene sequences or transcriptome profiles at a single cell level. Essentially, MOD allows for precise, flexible, scalable liquid handling that can build a large number of predetermined reaction conditions.

MOD not only allows for the sorting and combination of particulates (cells, beads, hydrogels, etc.), but also sort and assemble diverse droplet contents (e.g., antibody solutions, cell stains, oligonucleotides etc.). Furthermore, droplet experiments constructed with MOD are compartmentalized and miniaturized (e.g., ˜100 pL) providing contained reactions in concentrated volumes. These two aspects of MOD, reagent selection and reaction miniaturization, provide a powerful approach to phenotypically screen large numbers of single cells.

An indrop assay can be used to detect hybridomas secreting a desired antibody. Such assays may quantify antibody secreted by a hybridoma within individual droplets. In certain embodiments, the readout relies on a sandwich immunoassay, similar to ELISA, but in a “one-pot” format that is suitable for droplets and without washing steps.

Aptamer-based approaches typically rely on using a modified or unmodified nucleic acid that binds specifically to the target of interest (e.g., antibody secreted by a hybridoma, using antigen bait). Upon binding, the state, local environment, or structure of the aptamer changes allowing either direct detection, or amplification of the bound aptamer prior to detection. Binding of the aptamer to target can also release a hybridization partner (e.g. a complementary oligo or small molecule). Detection can be performed via fluorescence, absorbance, or quantification of the aptamer or released hybridization partner (see, e.g., Xue, L., et al. (2012). “Sensitive and homogeneous protein detection based on target-triggered aptamer hairpin switch and nicking enzyme assisted fluorescence signal amplification.” Anal Chem 84(8): 3507-3513; herein incorporated by reference).

Proximity assays rely on the interaction of two binding partners brought into close proximity by the target molecule (e.g., desired antibody and antigen bait). Binding partners are typically nucleic acids or protein fragments. Binding partners can be bound directly or indirectly to detection reagents, such as antibodies or aptamers, or bind the target directly. When multiple detection reagents (with interaction partners bound to them) are both bound to the target, or when multiple interaction partners bind the target directly, the close proximity of the attached binding partners allows for covalent linkage (e.g. via ligation), hybridization, or general interaction. The interaction of the binding partners allows for detection via quantification of the bound partners, amplification of bound partners, or direct measurement using for example fluorescence (See, e.g., Xiao, Q., et al. (2018). “Multiplexed chemiluminescence imaging assay of protein biomarkers using DNA microarray with proximity binding-induced hybridization chain reaction amplification.” Anal Chim Acta 1032: 130-137; herein incorporated by reference in its entirety). Fluorogenic and other activatable small-molecule detection strategies rely on direct modification of a substrate by the target of interest. Detection is performed directly on the modified substrate using for example fluorescence or absorbance.

In certain embodiments, the MOD platform is employed and droplet manipulation and sorting is be achieved by electrowetting, the modification of the wetting properties of a surface with an applied electric field. Electrowetting manipulation of droplets in a microfluidic device may be achieved through the application of differential voltages to different regions in an electrode grid (see, U.S. Pat. No. 6,911,132, herein incorporated by reference). Alternatively, droplet actuation and sorting can be achieved using opto-electrowetting, where localized electric fields are triggered through the selective application of light to a photoconductive layer (see, U.S. Pat. No. 6,958,132, which is herein incorporated by reference in its entirety).

In certain embodiments, droplet-based cell culture is performed using porous materials. The duration of cell culture in sub-nanoliter droplets is limited by a finite amount of encapsulated media and localized buildup of metabolic waste products. In cases where longer duration incubations are desired or required, it may be appropriate to convert a droplet to a media-permeable format while keeping encapsulated objects in place. This can be achieved by flowing hydrogel precursors into droplets along with cells, then triggering gelation to form either gel beads or permeable capsules. After gelation, the emulsion is broken, the emulsion oil is removed, and the hybridoma-laden beads or capsules are suspended in media and cultured for a time. Examples of the hydrogel bead approach are given in Wan et al., (Polymers (Basel)., vol. 4, no. 2, pp. 1084-1108, 2012), Utech et al., (Adv. Healthc. Mater., 2015), and Dolega et al. (Biomaterials, vol. 52, no. 1, pp. 347-357, 2015.)—all of which are herein incorporated by reference in their entireties. Examples of permeable capsules are given by Yu et al, (Biomed. Microdevices, vol. 17, no. 2, 2015.), van Loo et al (Mater. Today Bio, vol. 6, no. February, p. 100047, 2020.), and Leonaviciene et al. (Lab Chip, no. Advanced Article, 2020), all of which are herein incorporated by reference in their entireties. Extended cell culture is especially useful in cases where cell proliferation is important, such as clonal expansion of single cells and cell-cell interaction assays where proliferation is a readout. In some cases, it may be necessary to break down a gel bead or capsule via chemical, enzymatic, or thermal means in order to access the contents for further processing. In other cases, gel beads and capsules are sufficiently permeable that analysis can be performed with the materials in places, such as the washing in of assay reagents (Chokkalingam et al., Lab Chip, vol. 13, no. 24, pp. 4740-4744, 2013) or sequencing reagents (Leonaviciene).

Discrete entities as used or generated in connection with the subject methods, devices, and/or systems may be sphere shaped or they may have any other suitable shape, e.g., an ovular or oblong shape. Discrete entities may be droplets. Discrete entities as described herein may include a liquid phase and/or a solid phase material. In some embodiments, discrete entities according to the present disclosure include a gel material. In some embodiments, the subject discrete entities have a dimension, e.g., a diameter, of or about 1.0 μm to 1000 μm, inclusive, such as 1.0 μm to 750 μm, 1.0 μm to 500 μm, 1.0 μm to 100 μm, 1.0 μm to 10 μm, or 1.0 μm to 5 μm, inclusive. In some embodiments, discrete entities as described herein have a dimension, e.g., diameter, of or about 1.0 μm to 5 μm, 5 μm to 10 μm, 10 μm to 100 μm, 100 μm to 500 μm, 500 μm to 750 μm, or 750 μm to 1000 μm, inclusive. Furthermore, in some embodiments, discrete entities as described herein have a volume ranging from about 1 fL to 1 nL, inclusive, such as from 1 fL to 100 pL, 1 fL to 10 pL, 1 fL to 1 pL, 1 fL to 100 fL, or 1 fL to 10 fL, inclusive. In some embodiments, discrete entities as described herein have a volume of 1 fL to 10 fL, 10 fL to 100 fL, 100 fL to 1 pL, 1 pL to 10 pL, 10 pL to 100 pL or 100 pL to 1 nL, inclusive. In addition, discrete entities as described herein may have a size and/or shape such that they may be produced in, on, or by a microfluidic device and/or flowed from or applied by a microfluidic device.

In some embodiments, the discrete entities as described herein are droplets. The terms “drop,” “droplet,” and “microdroplet” are used interchangeably herein, to refer to small, generally spherically structures, containing at least a first fluid phase, such as an aqueous phase (e.g., water), bounded by a second fluid phase (e.g., oil) which is immiscible with the first fluid phase. In some embodiments, droplets according to the present disclosure may contain a first fluid phase (e.g., oil) bounded by a second immiscible fluid phase (e.g., an aqueous phase fluid, such as water). In some embodiments, the second fluid phase is an immiscible phase carrier fluid. Thus, droplets according to the present disclosure may be provided as aqueous-in-oil emulsions or oil in aqueous emulsions. Droplets may be sized and/or shaped as described herein for discrete entities. For example, droplets according to the present disclosure generally range from 1 μm to 1000 μm, inclusive, in diameter. Droplets according to the present disclosure may be used to encapsulate cells, nucleic acids (e.g., DNA), enzymes, reagents, and a variety of other components. The term droplet may be used to refer to a droplet produced in, on, or by a microfluidic device and/or flowed from or applied by a microfluidic device.

As used herein, the term “dielectrophoretic force” refers to the force exerted on an uncharged particle caused by of the polarization of the particle by and interaction with a nonuniform electric field. A dielectrophoretic force can be directed towards (i.e. “attractive dielectrophoretic force”), away from (i.e. “repulsive dielectrophoretic force,”) or in any direction relative to the source of the electric field. Before being contacted by the electric field, the particle can be positively charged, negatively charged, or neutral.

As used herein, the term “electrophoretic force” refers to the force exerted on a charged particle caused by interaction with an electric field. An electrophoretic force can be directed towards (i.e. “attractive electrophoretic force”,) away from (i.e. “repulsive electrophoretic force,”) or in any direction relative to the source of the electric field. Before being contacted by the electric field, the particle can be positively charged, negatively charged, or neutral.

As used herein, the term “carrier fluid” refers to a fluid configured or selected to contain one or more discrete entities (e.g., droplets) as described herein. A carrier fluid may include one or more substances and may have one or more properties (e.g., viscosity), which allow it to be flowed through a microfluidic device or a portion thereof. In some embodiments, carrier fluids include, for example: oil or water, and may be in a liquid or gas phase.

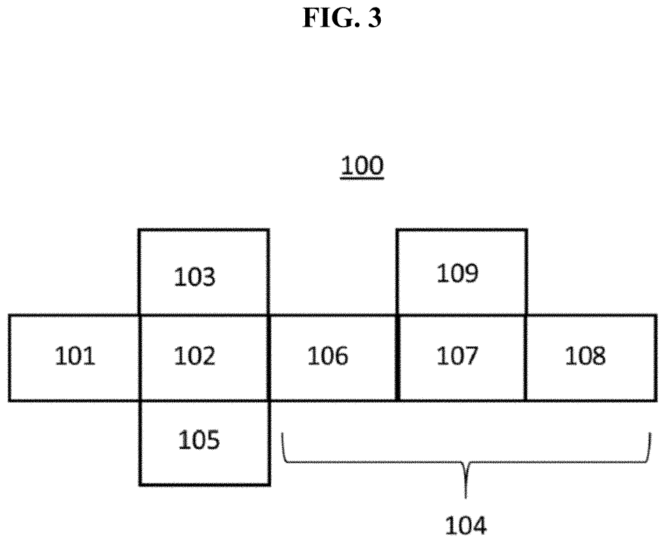

The present disclosure provides methods of selectively combining discrete entities using the MOD platform (e.g., to generate hybridomas from B-cells and myeloma cells). FIG. 3 presents a non-limiting, simplified, schematic representation of one type of device and method according to the present disclosure. The microfluidic device of FIG. 3 is labeled as microfluidic device 100. FIG. 3 shows a representation of an inlet channel 101, wherein a discrete entity that is insoluble and/or immiscible in a carrier fluid a carrier fluid can be flowed through the inlet channel 101 to a sorter channel 102 that is in direct fluid communication with inlet channel 101. Next, the discrete entity can be sorted into a first outlet channel 104 or a second outlet channel 105, which are both in direct fluid communication with the sorter channel, by sorting element 103. Sorting element 103 can be, in some cases, an electrode, such as an electrode that is configured to exert a dielectrophoretic force on the discrete entity. Sorting element 103 in FIG. 3 is configured to sort a discrete entity in sorting channel 102 to first outlet channel 104 or second outlet channel 105. In some cases, if the discrete entity is sorted to second outlet channel 105, the discrete entity is sorted to a waste container or is recycled back to inlet channel 101. FIG. 3 shows an embodiment wherein first outlet channel 104 includes an upstream region 106, a discrete entity merger region 107, and a downstream region 108. In some cases, the discrete entity merger region comprises a change in a dimension of the first outlet channel, such as where the discrete entity merger region 107 has a larger cross-sectional area than the upstream region 106.

In addition, the FIG. 3 device includes trapping element 109. In some cases, trapping element 109 includes a trapping electrode, and the trapping electrode is configured to exert a force (e.g. a dielectrophoretic force), that traps the discrete entity in the discrete entity merger region 107. Furthermore, the discrete entity merger region 107 and the trapping element 109 are configured such that a force applied by the trapping electrode in the discrete entity merger region is sufficient to trap a plurality of discrete entities in the discrete entity merger region for a time sufficient for the plurality of discrete entities to combine to form a combined discrete entity. In some cases, a trapping electrode is configured to provide an electric field that affects the surface of the discrete entities such that the discrete entities can more easily merge (e.g. the discrete entities will spontaneously merge). In some cases, the affecting is destabilizing.

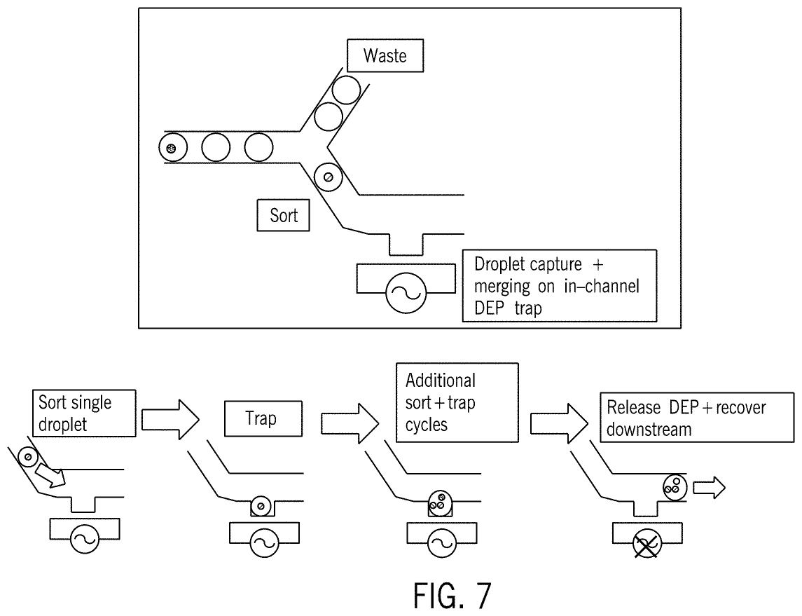

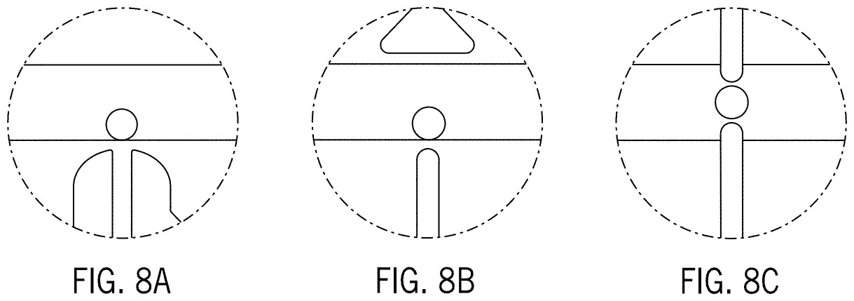

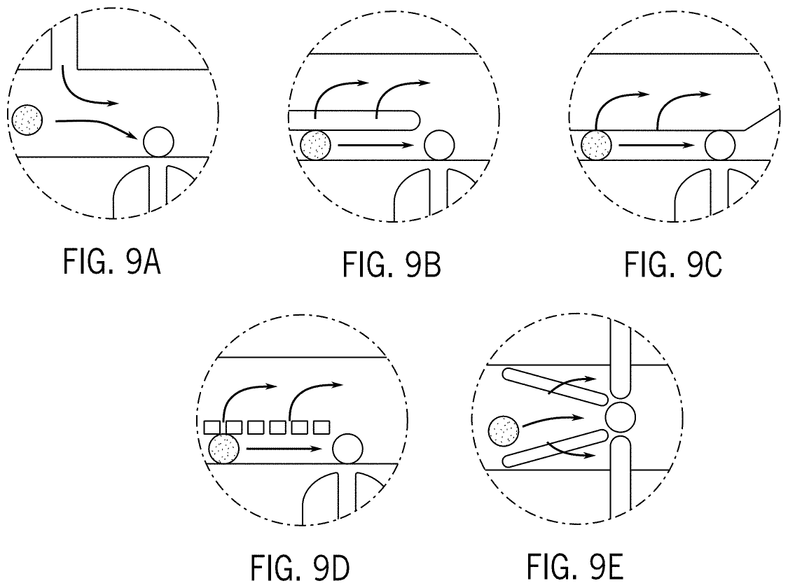

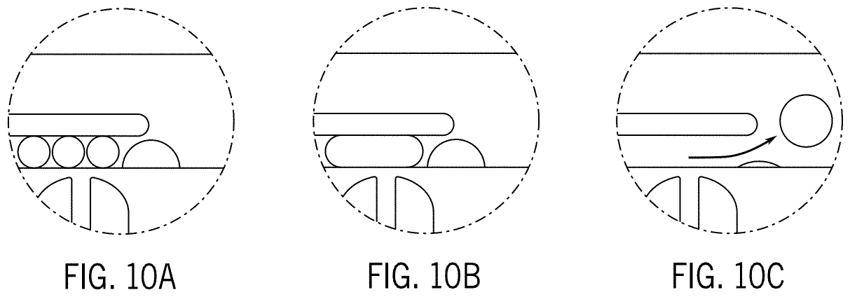

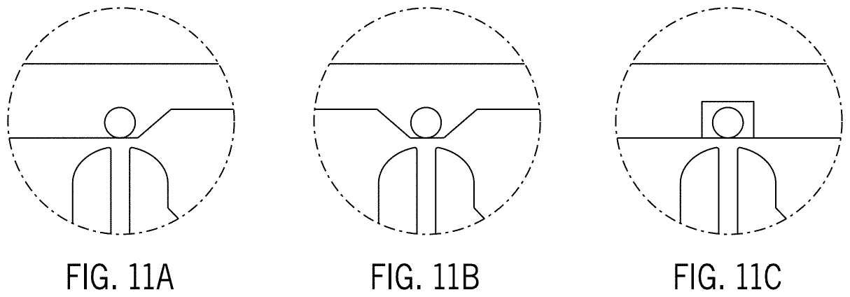

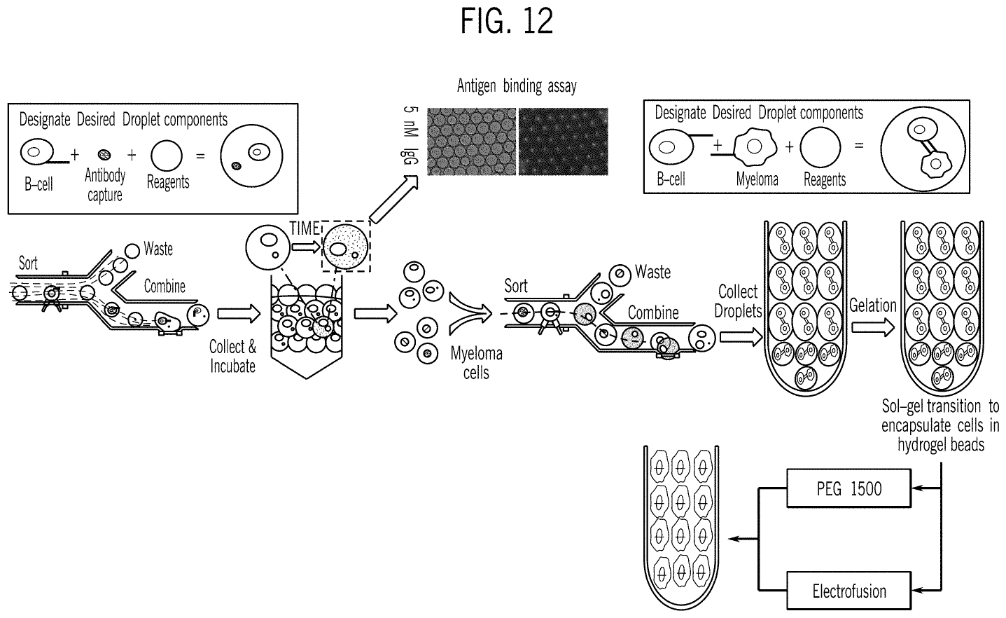

Methods of using the FIG. 3 device include flowing a plurality of discrete entities through inlet channel 101 to sorting channel 102, sorting with sorting element 103 the plurality of discrete entities into first outlet channel 104 or second outlet channel 105, trapping with trapping element 109 at least two discrete entities (e.g., one B-cell and one myeloma cell, each with cell surface tags that help bind the other cell) in discrete entity merger region 107 for a time sufficient for the at least two discrete entities to combine to form a combined discrete entity. FIG. 7 shows a schematic representation of an exemplary method wherein discrete entities containing cells are selectively combined.

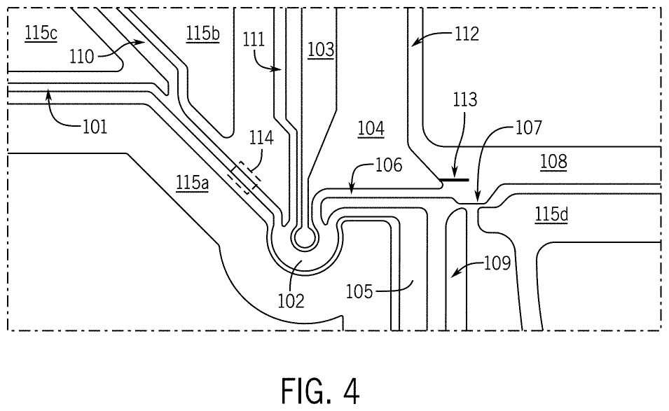

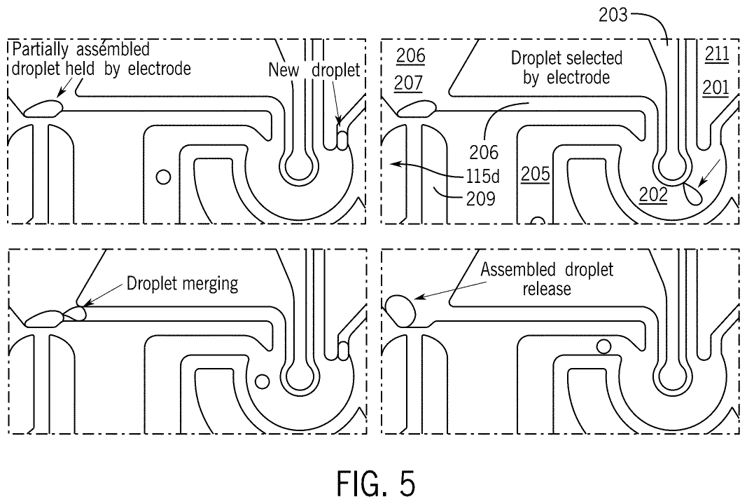

FIG. 4 presents an additional, non-limiting, simplified, schematic representation of one type of a device and method according to the present disclosure. In some cases, the discrete entity merger region includes a recess, such as shown as recess 107 in FIG. 4. In some cases, the discrete entity merger region includes a flow divider, such as shown as flow divider 113 in FIG. 4. In some cases, the device further includes a laminating oil inlet, such as shown as laminating oil inlet 112 in FIG. 4. In some cases, the trapping element includes two electrodes that have a significantly different shape from one another, such as shown as electrodes 109 in FIG. 4. In some cases, the trapping element includes two electrodes that produce a region of high electric field gradients that extends into the microfluidic channel. In some cases, the discrete entity merger region includes a change in the angle of flow between an adjacent upstream region and the discrete entity merger region, e.g. as shown in FIG. 5. In some cases, the device further includes a spacer fluid inlet. As an example, the device in FIG. 4 includes spacer fluid channel 110 in fluid communication with the inlet channel 101. The spacer fluid channel can be configured such that flowing spacer fluid through the spacer fluid channel causes spacer fluid to be located between two discrete entities flowing through the inlet channel, thereby maintaining or increasing the distance between the two discrete entities, thereby allowing each of the two discrete entities to be independently sorted or not sorted.

In some cases, the device further includes a bias fluid inlet. As an example, the device in FIG. 4 includes bias fluid channel 111 in fluid communication with sorter channel 102. The bias fluid channel can be configured such that flowing bias fluid through the bias fluid channel will cause a discrete entity to move closer to a second side wall of the sorter channel and farther away from a first side wall of the sorter channel. Thus, as an example, the spacer fluid inlet 111 would cause the discrete entity to move closer to the wall of the inlet channel that is closer to the bottom of the figure, and further away from the wall closer to the top of the figure. As such, one or more bias fluid channels can be configured such that a discrete entity will preferentially flow to a first outlet location or a second outlet location in the absence of a force from a sorting element. In some cases, the bias fluid inlet channel can be configured such that a discrete entity will preferentially flow to a second outlet channel in the absence of a dielectrophoretic force from a sorting electrode. As an example, the bias fluid inlet 111 in FIG. 4 causes a discrete entity to preferentially flow to second outlet channel 105 in the absence of a force exerted on the discrete entity by the sorting electrodes 103.

In some cases, the device includes a detector configured to detect a discrete entity in the input channel, wherein the microfluidic device is configured to sort a discrete entity in the sorting channel based on the detection by the detector. As an example, FIG. 4 shows an embodiment in which a discrete entity in detection region 114 of inlet channel 101 can be detected by a detector, after which sorting electrodes 103 can sort the discrete entity into the first outlet channel 104 or the second outlet channel 105. The FIG. 4 devices also includes shielding electrodes 115a, 115b, 115c, and 115d. As used herein, the term “shielding electrode” is used interchangeably with “moat electrode.” Each shielding electrode can be configured to perform one or more functions including: at least partially shielding discrete entities from undesired electromagnetic fields, assisting with the sorting of discrete entities, and assisting with the trapping of discrete entities.

As such, as used herein, shielding electrodes can also be referred to as sorting electrodes or trapping electrodes if such electrodes are configured to participate in the sorting or trapping of discrete entities. Hence, shielding electrode 115a can also be referred to as a sorting electrode if it is configured to form a bipolar electrode pair with sorting electrode 103 to facilitate the sorting of discrete entities. Similarly, shielding electrode 115d can also be referred to as a trapping electrode if it is configured to form a bipolar electrode pair with trapping electrode 109 to facilitate the trapping of discrete entities.

In some cases, a shielding electrode can generate an electromagnetic field such that discrete entities in the device is at least partially shielded from undesired electromagnetic fields. Such undesired electromagnetic fields can originate from outside the microfluidic device or from within the microfluidic device. In some cases, the undesired electromagnetic fields are those fields that are not generated by a sorting electrode or by a trapping electrode. By at least partially shielding discrete entities in the microfluidic device, the shielding electrodes can inhibit the unintended merging of discrete entities (i.e. merging of discrete entities outside the discrete entity merger region). In some cases, shielding electrodes 115a, 115b, and 115c can be used to at least partially shield discrete entities from electromagnetic fields that are not generated by the sorting electrode or the trapping electrode.

In some cases, shielding electrodes can assist with the sorting of discrete entities. As an example, shielding electrode 115a can interact with sorting electrode 103 in order to facilitate sorting, such as by forming a bipolar electrode pair with sorting electrode 103. In some cases, sorting electrode 103 can be the charged electrode (e.g. positively charged), and shielding electrode 115a can be a ground. Stated in another manner, shielding electrode 115a can be configured to influence the shape of the electromagnetic field generated by sorting electrode 103 in order to facilitate sorting.

In some cases, shielding electrodes can assist with the trapping of discrete entities. As an example, shielding electrode 115d can interact with trapping electrode 109 in order to facilitate trapping, such as by forming a bipolar electrode pair with trapping electrode 109. In some cases, sorting electrode 109 can be the charged electrode (e.g. positively charged), and shielding electrode 115d can be a ground. Stated in another manner, shielding electrode 115d can be configured to influence the shape of the electromagnetic field generated by trapping electrode 109 in order to facilitate sorting.

In some cases, one or more of the shielding electrodes are separate elements, such as when all the shielding electrodes are separate elements. In some cases, one or more of the shielding electrodes are directly electrically connected. In some cases, one or more of the shielding electrodes are different regions of a single electrode, such as part of a single piece of metal. In some cases, one or more of the shielding elements are attached to ground.

As shown in FIG. 4, in some cases, the device includes one or more shielding electrodes. In some cases, the device includes zero shielding electrodes, such as when the discrete entities are sorted using a single sorting electrode and the discrete entities are trapped using a single trapping electrode.

As such, discrete entities are sorted and selectively combined within a microfluidic device (i.e., without leaving the microfluidic device). Stated in another manner, the discrete entities are sorted and combined without leaving microfluidic sized channels and regions.

In addition, the present disclosure provides examples of specific elements and steps that can be used with the described devices, systems, and methods. As reviewed above, the trapping element and the sorting element can be electrodes that exert a dielectrophoretic force on the discrete entity. In some cases, the electrodes are microfluidic channels containing a conductive material (e.g. salt water, liquid metal, molten solder, or a conductive ink to be annealed later). In some cases, the electrodes are patterned on the substrate of the microfluidic device (e.g. a patterned indium tin oxide (ITO) glass slide). In some cases, the trapping element includes two electrodes. In some cases, the trapping element is a selectively actuatable bipolar droplet trapping electrode. In some cases, the sorting element includes two electrodes. In some cases, the sorting element includes a selectively actuatable bipolar droplet sorting electrode.

In some cases, the sorting channel includes a partial height flow divider. In some cases, the sorting channel has a concentric or essentially concentric flow path and a portion of the sorting electrode is positioned at the center of the arc of the concentric or essentially concentric flow path.

In some embodiments, the discrete entity includes a particle (e.g. a cell, such as a B-cell or myeloma). In some embodiments, the discrete entity includes a chemical reagent (e.g. a lysing agent or a PCR reagent). In some embodiments, the discrete entity includes both a cell and a chemical reagent. In some embodiments, the discrete entity includes a fluorescently tagged B-cell or myeloma.

In some cases, the sorting is passive sorting. In some cases, the sorting is active sorting (i.e., the sorting element sorts a discrete entity into one of at least two locations based on a detected property of the discrete entity or a component within the discrete entity). In some cases, the detected property is an optical property and the device further includes an optical detector (e.g. an optical detector configured to detect an optical property of a discrete entity or a component within in the inlet channel). In some cases, the optical property is fluorescence and the device further includes a source of excitation light. In some cases, the sorting is based on the detected fluorescence of a fluorescent tag on a cell in the discrete entity.

In some cases, the discrete entity merger region can include structural elements that are configured to aid in the trapping and combination of discrete entities therein. In some cases, such structural elements are configured to aid in such trapping and combining by changing the speed or direction of the flow of fluid through an area of the discrete entity merger region.

The present disclosure also provides methods of using systems that include a microfluidic device, e.g. as described above, and one or more additional components, e.g. (a) a temperature control module operably connected to the microfluidic device; (b) a detector configured to detect a discrete entity in the input channel, wherein the microfluidic device is configured to sort a discrete entity in the sorting channel based on the detection by the detector; (c) an incubator operably connected to the microfluidic device or a discrete entity maker; (d) a sequencer operably connected to the microfluidic device; (e) a device configured to make a plurality of discrete entities, wherein the device is located within the microfluidic device or separately from the microfluidic device; and (f) one or more conveyors configured to convey a particle (e.g. a cell, or a discrete entity), wherein the discrete entity can contain a particle in some cases, between any combination of: the incubator, device configured to make a plurality of discrete entities, the microfluidic device, the sequencer.

In some cases, the methods include controlling the temperature of the microfluidic device using a temperature control module operably connected to the microfluidic device. In some cases, the methods include detecting a discrete entity in the input channel of the microfluidic device (e.g. detecting an optical property of the discrete entity or a component therein), and sorting the discrete entity based on the detecting. In some cases, the method includes incubating cells in an incubator that is operably connected to discrete entity maker or a microfluidic device. In some cases, the method includes making discrete entities with a discrete entity maker, wherein the discrete entity maker is located within the microfluidic device or separate from the microfluidic device. In some cases, the method includes moving a discrete entity between components of the system (e.g. with one or more conveyors).

The present disclosure also provides steps that can be performed after the release of a combined microfluidic droplet from a discrete entity merger region. In some cases, the method includes recovering a component (e.g. a cell, a chemical compound or a combination thereof), from the combined discrete entity. In cases where a combined discrete entity includes one or more cells, the one or more cells can be analyzed (e.g. genetic information therein, such as the sequence of the monoclonal antibody in the hybridoma) can be sequenced using a sequencer. The genetic information can include, e.g. DNA and RNA. In some cases, the sequencing includes PCR. In some cases, the analysis of a discrete entity can include mass spectrometry. In some cases, the method includes printing the combined discrete entity onto a substrate, e.g. as described in US 2018/0056288, which is incorporated herein by reference for its disclosure of printing a discrete entity onto a substrate.

The present disclosure also provides a method of selectively performing reactions by selectively combining two or more discrete entities (e.g., each containing a B-cell or myeloma cell), as described above, wherein the reaction occurs between one or more components from each discrete entity. Such components can be one or more cells, one or more products derived from a cell, one or more reagents, or a combination thereof. In some cases, the one or more products derived from a cell include cell lysate, DNA, RNA, or a combination thereof. As an example, FIGS. 6A-H show the combination of four discrete entities, wherein three of the discrete entities each contain a different reagent and the fourth discrete entity contains a single cell (e.g., myeloma or B-cell). As such, FIGS. 6A-H show that a microfluidic device as described herein can be used to selectively combine different discrete entities, resulting in the formation of a combined discrete entity (e.g., that contains the three reagents and the B-cell and/or myeloma cell). In other embodiments, four discrete entities are employed, where one droplet contains a B-cell, one droplet contains a myeloma cell, one contains alginate (for forming hydrogel), and one contains Ca2+ ions (for triggering formation of hydrogel when mixed with alginate).

TABLE 1

| Droplet 1 | Droplet 2 | Droplet 3 | Droplet 4 |
|---|---|---|---|
| B-cell | Alginate | Myeloma | Ca²⁺ |
| Alginate | B-cell | Myeloma | Ca²⁺ |
| Myeloma | B-cell | Alginate | Ca²⁺ |
| Myeloma | Alginate | B-cell | Ca²⁺ |
| B-cell | Myeloma | Alginate | Ca²⁺ |
| Alginate | Myeloma | B-cell | Ca²⁺ |

As such, the method of selectively performing reactions can include the combination of two or more discrete entities (e.g. three or more and four or more), which allows a B-cell and myeloma cell to be brought together. In some cases, the number of discrete entities that contain at least one cell is zero discrete entities, one discrete entity, two discrete entities, or three or more discrete entities. In some cases, the number of cells in a discrete entity is one. In some cases, the method includes repeating the selective combination of discrete entities (e.g. performing the selective combination two or more times, three or more times, or four or more times, or 1000 or more times or a million or more times).

The present methods allow for the selective combination of two or more discrete entities without the need to accurately time the release or to accurate time the sorting of the two or more discrete entities. As such, in some cases, a first discrete entity is trapped in the discrete entity merger region before a second discrete entity to be combined therewith has entered the outlet channel after being sorted. In some cases, the second discrete entity has not entered the sorter channel, has not entered the inlet channel, or has not even been made when the first discrete entity is trapped in the discrete entity merger region.

The present methods allow for the sorting of discrete entities based on whether they contain a B-cell or myeloma cell, and allows the selective combination of only those discrete entities that contain the desired components. In some cases, the method involves creating 5 or more combined discrete entities per minute, including 10 or more, 25 or more, 50 or more, 75 or more, 100 or more, 150 or more, 200 or more, or 300 or more. In some cases, the method involves making 300 or more combined discrete entities per hour, including 1,500 or more, 3,000 or more, 4,500 or more, 6,000 or more, 9,000 or more, 12,000 or more, or 21,000 or more. In some cases, the sorting step is performed such that discrete entities are sorted at a rate of 0.01 Hz or more (e.g. 0.1 Hz or more, 1 Hz or more, 10 Hz or more, 100 Hz or more, 1 kHz or more, 10 kHz or more, or 30 kHz or more). In some cases, an electromagnetic sorter is used instead of a mechanical sorter (e.g. a valve, to allow for faster sorting rates). In some cases, the trapping and combining steps are performed such that a combined discrete entity is formed or released at a rate of 1 Hz or more, e.g. 10 Hz or more, 100 Hz or more, or 1,000 Hz or more.

In some cases, a discrete entity is flowed such that it reaches the discrete entity merger region between 0.1 ms to 1,000 ms after being sorted, such as between 1 ms and 100 ms, between 2 ms and 50 ms, and between 5 ms and 25 ms. In some cases, the first outlet channel is between 0.2 mm long and 5 mm long. In some cases, the first outlet channel has a dimension (e.g., width or height or diameter) of between 5 μm and 500 μm, such as between 10 μm and 100 μm. In some cases, the carrier fluid containing the discrete entities is flowed into the inlet channel at a rate of between 1 μl per hour and 10,000 μl per hour, such as between 10 μl per hour and 1,000 μl per hour, 25 μl per hour and 500 μl per hour, and between 50 μl per hour and 250 μl per hour. In some cases, the spacer fluid is injected at a rate of between 100 μl per hour and 20,000 μl per hour, such as 500 μl per hour and 5,000 μl per hour. In some cases, the bias fluid is injected at a rate of between 100 μl per hour and 20,000 μl per hour, such as 500 μl per hour and 5,000 μl per hour. In some cases, the fluid used to create cell-containing discrete entities has a concentration of between 1,000 cells per ml and 10,000,000 cells per ml, such as between 10,000 cells per ml and 1,000,000 cells per ml, and between 50,000 cells per ml and 200,000 cells per ml. In some cases, the discrete entities have a volume between 1 pl and 10,000 pl, such as between 10 pl and 1,000 pl, or between 50 pl and 500 pl.

In some cases, the one or more cells from a combined discrete entity are cultured for at least 30 minutes or more, such as 1 hour or more, 6 hours or more, 12 hours or more, 24 hours or more, 3 days or more, or 7 days or more. In some cases, the device can continuously operate by selectively combining discrete entities for 10 minutes or more, such as 30 minutes or more, 45 minutes or more, 90 minutes or more, or 180 minutes or more. In some cases, the device can make at least 100 combined discrete entities while continuously operating, such as 1,000 combined discrete entities or more, 10,000 combined discrete entities or more, or 100,000 combined discrete entities or more.

In some cases, the methods include making one or more discrete entities, such as with a discrete entity maker. In such cases, the discrete entity maker can be part of the microfluidic device or separate from the microfluidic device as otherwise described herein. If the discrete entity maker is separate from the microfluidic device, the discrete entity maker can be operably connected to the microfluidic device (e.g., such that discrete entities can flow from the maker to the microfluidic device), or the discrete entities can be moved to the microfluidic device without the discrete entity maker and microfluidic device being operably connected. The systems and devices can include one or more discrete entity makers configured to form discrete entities from a fluid stream. Suitable discrete entity makers include selectively activatable droplet makers and the methods may include forming one or more discrete entities via selective activation of the droplet maker. The methods may also include forming discrete entities using a droplet maker, wherein the discrete entities include one or more entities which differ in composition. In some cases, the discrete entity maker comprises a T-junction and the method includes T-junction drop-making. In some cases, making the discrete entities includes a step of emulsification. In some cases, the discrete entity maker is made, in part or in whole, of a polymer. In some cases, one or more surfaces of the discrete entity maker are coated with a fluorosilane (e.g. such a discrete entity maker can be used when fluorinated fluids are passed through the discrete entity maker).

In some cases when multiple types of discrete entities are made (e.g., discrete entities that contain different contents, such as one with a B-cell and one with an myeloma cell), the contents can affect the ability of the discrete entity maker to successfully make the discrete entities. As such, in some cases, different conditions for the discrete entity maker are used to make a first group of discrete entities with first contents than for making a second group of discrete entities with second contents.

Aspects of the disclosed methods may include making discrete entities using one or more cells from a biological sample. In such cases, each discrete entity may contain zero, one, or more than one cell. In some cases, such discrete entities can be made by incorporating the biological sample, cells from the biological sample, lysate from cells of the biological sample, or any other sample derived from the biological sample into a mixed emulsion. In some cases, the method further includes separating one or more components of the biological sample or otherwise processing the biological sample (e.g. via centrifugation, filtration, and the like), before making the discrete entities.

In some cases, after the making of the discrete entities but before introducing the discrete entities to an inlet channel of a microfluidic device as described herein, the discrete entities can be further modified (e.g. by adding a B-cell, myeloma cell, a reagent, a drug, a hydrogel, an extracellular matrix, a bead, a particle, a biological material, media, or a combination thereof). In some cases, the reagent is a primer, a probe, a lysing agent, a surfactant, a detergent, a barcode, or a fluorescent tag. In some cases, the bead is an RNA capture bead. In some cases, the bead is an immunoassay bead. In some cases, the barcode is an oligonucleotide. In some cases, different types of discrete entities are labeled with different types of barcodes, fluorescent tags, or a combination thereof.

Fluorescent tags can be used to image a discrete entity or combined discrete entity in the discrete entity merger region. Fluorescent tags can also be used to identify the particular type of discrete entities that were combined to create a given combined discrete entity. As such, the properties of the combined discrete entity or component thereof can be correlated with the contents that were used to make the original discrete entities. As an example, different types of B-cells can be labeled with different fluorescent tags and incorporated into discrete entities. After such B-cell-containing discrete entities are combined with other discrete entities (e.g. containing myeloma cells), the outcome of the combined discrete entities can be observed. As some of all of the original discrete entities can be labeled with fluorescent tags, the resulting combined discrete entity can have multiple fluorescent tags. In other cases, the combined discrete entity only has one fluorescent tag. Oligonucleotide barcodes can be used in a similar manner to that of fluorescent tags. Instead of detecting optical fluorescence, however, the oligonucleotide barcodes can be sequenced in order to identify the original discrete entities that formed the combined discrete entity.

Methods and devices which may be utilized in the encapsulating of a component from a biological sample are described in PCT Publication No. WO 2014/028378, the disclosure of which is incorporated by reference herein in its entirety and for all purposes. Encapsulation approaches of interest also include, but are not limited to, hydrodynamically-triggered drop formation and those described by Link, et al., Phys. Rev. Lett. 92, 054503 (2004), the disclosure of which is incorporated herein by reference. Other methods of encapsulating cells into droplets may also be applied. Where desired, the cells may be stained with one or more antibodies and/or probes prior to encapsulating them into drops.

One or more lysing agents may also be added to the discrete entities (e.g., droplets), containing a cell, under conditions in which the cell(s) may be caused to burst, thereby releasing their genomes. The lysing agents may be added after the cells are encapsulated into discrete entities. Any convenient lysing agent may be employed, such as proteinase K or cytotoxins. In particular embodiments, cells may be co-encapsulated in drops with lysis buffer containing detergents such as Triton X100 and/or proteinase K. The specific conditions in which the cell(s) may be caused to burst will vary depending on the specific lysing agent used. For example, if proteinase K is incorporated as a lysing agent, the discrete entities (e.g., droplets), may be heated to about 37-60° C. for about 20 min to lyse the cells and to allow the proteinase K to digest cellular proteins, after which they may be heated to about 95° C. for about 5-10 min to deactivate the proteinase K. In certain aspects, cell lysis may also, or instead, rely on techniques that do not involve addition of lysing agent. For example, lysis may be achieved by mechanical techniques that may employ various geometric features to effect piercing, shearing, abrading, etc. of cells. Other types of mechanical breakage such as acoustic techniques may also be used. Further, thermal energy can also be used to lyse cells. Any convenient methods of effecting cell lysis may be employed in the methods described herein as appropriate.

One or more primers may be introduced into the discrete entities for each of the genes to be detected. Hence, in certain aspects, primers for all target genes (e.g., antibody genes) may be present in the discrete entity at the same time, thereby providing a multiplexed assay. The discrete entities may be temperature-cycled so that discrete entities will undergo PCR. In certain embodiments, rolling circle amplification (RCA)-based proximity ligation is employed.

In some embodiments, a surfactant may be used to stabilize the discrete entities. In some cases, the discrete entities or the associated emulsion lack a surfactant. Accordingly, a discrete entity may involve a surfactant stabilized emulsion. Any convenient surfactant that allows for the desired reactions to be performed in the discrete entities, may be used. In other aspects, a discrete entity is not stabilized by surfactants or particles. The surfactant used depends on a number of factors such as the oil and aqueous phases (or other suitable immiscible phases (e.g., any suitable hydrophobic and hydrophilic phases)) used for the emulsions. For example, when using aqueous droplets in a fluorocarbon oil, the surfactant may have a hydrophilic block (PEG-PPO) and a hydrophobic fluorinated block (Krytox® FSH). If, however, the oil was switched to be a hydrocarbon oil, for example, the surfactant would instead be chosen so that it had a hydrophobic hydrocarbon block, like the surfactant ABIL EM90. In selecting a surfactant, desirable properties that may be considered in choosing the surfactant may include one or more of the following: (1) the surfactant has low viscosity; (2) the surfactant is immiscible with the polymer used to construct the device, and thus it doesn't swell the device; (3) biocompatibility; (4) the assay reagents are not soluble in the surfactant; (5) the surfactant exhibits favorable gas solubility, in that it allows gases to come in and out; (6) the surfactant has a boiling point higher than the temperature used for PCR (e.g., 95° C.); (7) the emulsion stability; (8) that the surfactant stabilizes drops of the desired size; (9) that the surfactant is soluble in the carrier phase and not in the droplet phase; (10) that the surfactant has limited fluorescence properties; and (11) that the surfactant remains soluble in the carrier phase over a range of temperatures. Other surfactants can also be envisioned, including ionic surfactants. Other additives can also be included in the oil to stabilize the discrete entities including polymers that increase discrete entity stability at temperatures above 35° C.

The discrete entities (e.g., microdroplets) described herein may be prepared as emulsions, such as an aqueous phase fluid dispersed in an immiscible phase carrier fluid (e.g., a fluorocarbon oil or a hydrocarbon oil) or vice versa. In some cases, the carrier fluid comprises a fluorinated compound. In some cases, the carrier fluid is an aqueous fluid. The nature of the microfluidic channel (or a coating thereon) (e.g., hydrophilic or hydrophobic), may be selected so as to be compatible with the type of emulsion being utilized at a particular point in a microfluidic workflow.

Emulsions may be generated using microfluidic devices. Microfluidic devices can form emulsions composed of droplets that are uniform in size. The microdroplet generation process may be accomplished by pumping two immiscible fluids, such as oil and water, into a junction. The junction shape, fluid properties (viscosity, interfacial tension, etc.), and flow rates influence the properties of the microdroplets generated but, for a relatively wide range of properties, microdroplets of controlled, uniform size can be generated using methods like T-junctions and flow focusing. To vary microdroplet size, the flow rates of the immiscible liquids may be varied since, for T-junction and flow focus methodologies over a certain range of properties, microdroplet size depends on total flow rate and the ratio of the two fluid flow rates. To generate an emulsion with microfluidic methods, the two fluids are normally loaded into two inlet reservoirs (syringes, pressure tubes) and then pressurized as needed to generate the desired flow rates (using syringe pumps, pressure regulators, gravity, etc.). This pumps the fluids through the device at the desired flow rates, thus generating microdroplet of the desired size and rate.

In some cases, a cell in a discrete entity may be labeled (e.g., by a fluorescent label, a barcode, or a combination thereof). In practicing the subject methods, a number of reagents may be incorporated into and/or encapsulated by, the discrete entities in one or more steps (e.g., about 2, about 3, about 4, or about 5 or more steps). Such reagents may include, for example, amplification reagents, such as Polymerase Chain Reaction (PCR) reagents. The methods of adding reagents to the discrete entities may vary in a number of ways. Approaches of interest include, but are not limited to, those described by Ahn, et al., Appl. Phys. Lett. 88, 264105 (2006); Priest, et al., Appl. Phys. Lett. 89, 134101 (2006); Abate, et al., PNAS, Nov. 9, 2010 vol. 107 no. 45 19163-19166; and Song, et al. Anal. Chem., 2006, 78 (14), pp 4839-4849; the disclosures of which are incorporated herein by reference. For instance, a reagent may be added to a discrete entity by a method involving merging a discrete entity with a second discrete entity which contains the reagent(s) in a discrete entity merger region of a microfluidic device described herein.

In certain embodiments, overlap extension PCR (OE-PCR) is employed to both amplify nucleic acid from cells (e.g., hybridomas). For example, after imaging a droplet and find a hybridoma, such cells could be lysed releasing the nucleic acid. To such droplets, Drop-seq beads and RT-PCR reagents are added to capture the mRNAs on the Drop-seq bead. The PCR could use overlap extension PCR to associate the variable region nucleic acid with the nucleic acid. Details on overlap extension PCR are found in the art, including at, for example, U.S. Pat. Pub. 20150154352 and Turchaninova et al., Eur. J. Immunol. 2013. 43: 2507-2515, both of which are herein incorporated by reference.

One or more reagents may also, or instead, be added using techniques such as droplet coalescence, or picoinjection. In droplet coalescence, a target drop may be flowed alongside a microdroplet containing the reagent(s) to be added to the droplet. The two droplets may be flowed such that they are in contact with each other, but not touching other microdroplets. These drops may then be passed through electrodes or other aspects for applying an electrical field, wherein the electric field may destabilize the microdroplets such that they are merged together. Reagents may also, or instead, be added using picoinjection. In this approach, a target drop may be flowed past a channel containing the reagent(s) to be added, wherein the reagent(s) are at an elevated pressure. Due to the presence of the surfactants, however, in the absence of an electric field, the microdroplet will flow past without being injected, because surfactants coating the microdroplet may prevent the fluid(s) from entering. However, if an electric field is applied to the microdroplet as it passes the injector, fluid containing the reagent(s) will be injected into the microdroplet. The amount of reagent added to the microdroplet may be controlled by several different parameters, such as by adjusting the injection pressure and the velocity of the flowing drops, by switching the electric field on and off, and the like.

In some cases, a discrete entity includes a bead. In some cases, at least one dimension of the bead (e.g., diameter, is between about 0.5 μm and about 500 μm). In some cases, the bead is made of a polymeric material, such as polystyrene. In some cases, the bead is magnetic or contains a magnetic component. In some cases, the bead has a biomolecule attached to its surface, such as an antibody, a protein, an antigen, DNA, RNA, streptavidin, or a combination thereof. In some cases, the bead is an immunoassay bead. In some cases, the bead is an RNA capture bead. As such, the present disclosure provides methods of selectively combining a biomolecule with another compound or cell, wherein the method includes selectively isolating the biomolecule from a composition using the bead, making a discrete entity that includes the bead and biomolecule, and selectively combining the discrete entity containing the bead and biomolecule with one or more other discrete entities that contain one or more other compounds or cells using the microfluidic device described herein. Methods of selectively isolating biomolecules using beads are known in the art, e.g. U.S. 2010/0009383, which is incorporated herein by reference for its disclosure of a method of separating a biomolecule or cell using beads.

In some embodiments, the methods, devices, and/or systems described herein can be used to sequence nucleic acid derived from single cells (e.g., hybridomas). For example, individual cells can be encapsulated in the droplets and dispensed to the substrate as described herein. The cells can then be lysed and subjected to molecular biological processing to amplify and/or tag their nucleic acids with barcodes. The material from all the droplets can then be pooled for all cells and sequenced and the barcodes used to sort the sequences according to single droplets or cells. These methods can be used, for example, to sequence the genomes or transcriptomes of single cells in a massively parallel format.

As described above, in certain embodiments, nucleic acid sequence assay components that employ barcoding for labelling individual mRNA molecules, and/or for labeling for cell/well source (e.g., if wells pooled before sequencing analysis), and/or for labeling particular affixed entities (e.g., if droplet from two or more affixed entities are pooled prior to sequencing) are employed. Examples of such barcoding methodologies and reagents are found in Pat. Pub. US2007/0020640, Pat. Pub. 2012/0010091, U.S. Pat. Nos. 8,835,358, 8,481,292, Qiu et al. (Plant. Physiol., 133, 475-481, 2003), Parameswaran et al. (Nucleic Acids Res. 2007 October; 35(19): e130), Craig et al. reference (Nat. Methods, 2008, October, 5(10):887-893), Bontoux et al. (Lab Chip, 2008, 8:443-450), Esumi et al. (Neuro. Res., 2008, 60:439-451), Hug et al., J. Theor., Biol., 2003, 221:615-624), Sutcliffe et al. (PNAS, 97(5):1976-1981; 2000), Hollas and Schuler (Lecture Notes in Computer Science Volume 2812, 2003, pp 55-62), and WO201420127; all of which are herein incorporated by reference in their entireties, including for reaction conditions and reagents related to barcoding and sequencing of nucleic acids.

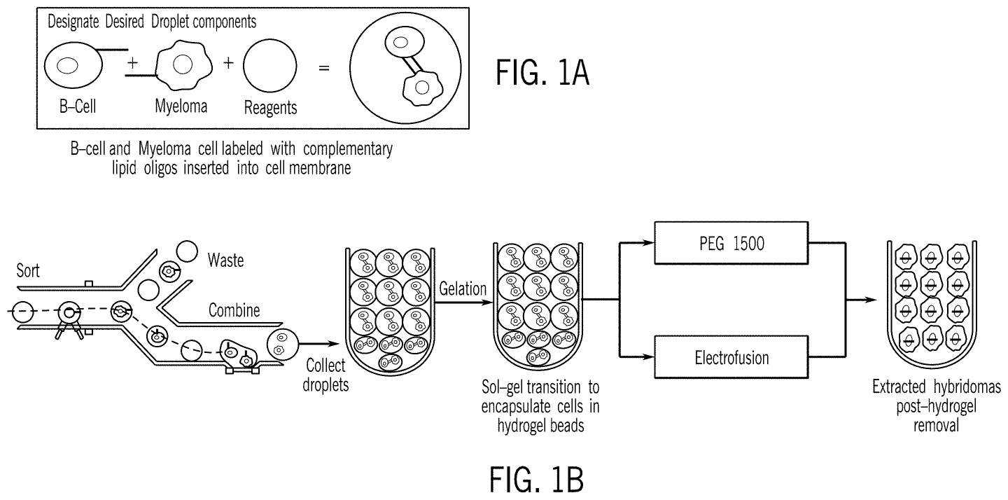

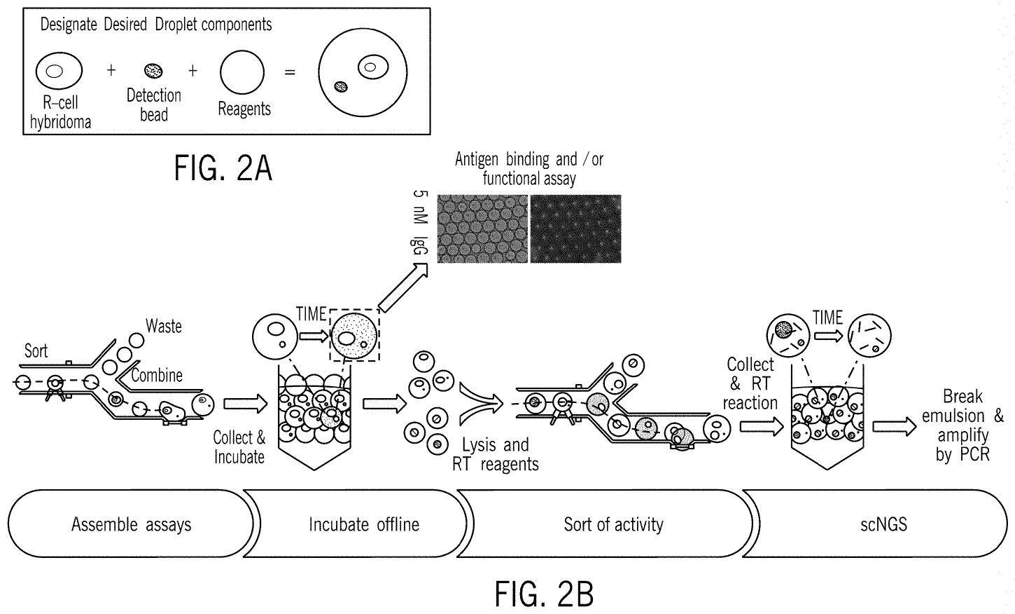

In certain embodiments, the DropSeq method employing beads with primers attached to them are employed to sequence nucleic acids from hybridomas. An example of such a method is described in Macosko et al., Cell, 161(5):1202-1214 (see, e.g., FIG. 1), which is herein incorporated by reference in its entirety. In certain embodiments employing DropSeq, barcoded template switch oligos are bound to beads and oligo dT is supplied in solution along with RT PCR reagents. Reverse transcription (RT) can, for example, be performed as described in Kim et al., Anal Chem. 2018 Jan. 16; 90(2):1273-1279, herein incorporated by reference. In other embodiments, barcoded oligo-dT beads are provided, the cells are lysed, mRNAs is captured on the beads, the emulsion is broken, and the drop is re-emulsified to capture mRNA beads with barcoded TSO beads where the TSO can be released by UV. Solution phase TSO can then be used for performing RT-PCR. Primers specific to the variable regions displayed on the surface of the SD cells can be employed to amplify such variable regions prior to sequencing.

In certain embodiments, unique oligo drops are provided to the fixed entities, and allow a link between imaging and genomics. For example, the unique oligos can contain two part 8 mer barcodes linked to polyA or TSO followed by 8-mer barcodes. In this regard, if one employs 96 barcoded oligos, selecting any three can generate 142,880 combinations. It is known what combination of three oligos are printed at each well position to identify that particular well. These oligos will also be sequenced and so when one sees a particular 3-oligo combination in the sequencing readouts, one knows the fixed entity and the image for that fixed entity.

In certain embodiments, the barcode tagging and sequencing methods of WO2014201273 (“SCRB-seq” method, herein incorporated by reference) are employed. The necessary reagents for the SCRB-seq method (e.g., modified as necessary for small volumes) are added to the fixed entities, each containing a lysed cell (e.g., hybridoma cell). Briefly, the SCRB-seq method amplifies an initial mRNA sample from cells from a single fixed entity. Initial cDNA synthesis uses a first primer with: i) N6 for cell/well identification, ii) N10 for particular molecule identification, iii) a poly T stretch to bind mRNA, and iv) a region that creates a region where a second template-switching primer will hybridize. The second primer is a template switching primer with a poly G 3′ end, and 5′ end that has iso-bases. After cDNA amplification, the tagged cDNA single fixed entity samples are pooled. Then full-length cDNA synthesis occurs with two different primers, and full-length cDNA is purified. Next, a NEXTERA sequencing library is prepared using an i7 primer (adds one of 12 i7 tags to identify particular multi-well plates) and P5NEXTPT5 to add P5 tag for NEXTERA sequencing (P7 tag added to the other end for NEXTERA). The library is purified on a gel, and then NEXTERA sequencing occurs. As a non-liming example, with twelve i7 plate tags, and 384 cell/well-specific barcodes, this allows total of 4,608 single cell transciptomes to be done at once. This method allows for quantification of mRNA transcripts in single fixed entity.

In other embodiments, the barcode tagging and sequencing methods employ concepts from the Multi-seq method. For example, cells are incubated with anchor and co-anchor lipid modified oligonucleotides (LMO) and encapsulated in droplets. Individual barcodes in droplets can hybridize to exposed regions of the LMOs and these barcodes can be used instead of Drop-seq beads. Anchor-coanchor LMOs remain bound to individual cells at 4° C. but can freely equilibrate between cells in a droplet at 37° C. Thus, a specific LMO-barcode combination in each droplet can be used to link two cells in that droplet that can be tracked after emulsion breaking. In one example, a unique LMO-barcode combination can be randomly assembled in every microfluidic droplet. Barcodes may also be deterministically pre-printed to a microwell array, and additionally provide linkage to imaging data recoded at specific microwell positions. In another embodiment, one cell in each combination may be LMO-barcoded before the combination in droplets. During incubation at 37° C., the LMO-barcodes will re-equilibrate to the initially non-barcoded cell and provide lasting information about co-encapsulation. If a unbarcoded B-cell is combined with an LMO-barcoded antigen presenting cell (APC), this process will allow the type of APC to be read out by sequencing only the B-cell.

In practicing the methods of the present disclosure, one or more sorting steps may be employed. A sorting step sorts a discrete entity into one of two or more locations (e.g. into one of two or more fluid channels). In some cases, the sorting is into one of two fluid channels. Discrete entities are sorted based on one or more properties of the discrete entity or a component within the discrete entity. In addition, such sorting may either be passive sorting or active sorting. Active sorting includes the detection of one or more properties of a discrete entity, or a component within the discrete entity, and sorting based on the detected property. Passive sorting involves sorting a discrete entity without the active detection of a property. Sorting approaches of interest include, by are not necessarily limited to, approaches that involve the use of one or more sorting channels and one or more sorting elements.

Sorting approaches which may be utilized in connection with the disclosed methods, systems and devices also include those described herein, and those described by Agresti, et al., PNAS vol. 107, no 9, 4004-4009. For active sorting, the device includes one or more sorting elements and one or more detectors, wherein each detector is configured to detect one or more properties of a discrete entity, or a component within the discrete entity, and each sorting element is configured to sort the discrete entity into one of two or more locations based on the detecting by the detection element. In some cases, a sorting element is positioned in proximity to the sorting channel, such as an electrode in proximity to the sorting channel. In some cases, a sorting element is positioned within the sorting channel, such as a partial height flow divider in a sorting channel. In some cases, the device includes a sorting element positioned within the sorting channel and one or more sorting elements positioned in proximity to the sorting channel. Exemplary structures and methods for active sorting discrete entities are described in Cole et al., PNAS, 2017, 114, 33, 8728-8733; Clark et al., Lab Chip, 2018, 5, 18, 710-713; and Sciambi et al., Lab on a Chip, 2015, 15, 47-51, the disclosures of which are incorporated herein by reference for sorting elements.

In some cases, the sorting element comprises an electrode configured to exert a dielectrophoretic force, an electrode configured to exert an electrophoretic force, an element configured to exert an acoustic force, a valve, or a combination thereof. In some cases, a sorting element comprises an electrode that is positioned in proximity to the sorting channel, e.g. an electrode configured to exert a dielectrophoretic force on the discrete entity or an electrophoretic force on the discrete entity. In some cases, the electrode is configured to exert an electrophoretic force on the discrete entity. The dielectrophoretic force on the discrete entity can be directed towards the electrode. In some cases, the sorting electrode is a liquid electrode, such as a microfluidic channel containing a conductive material, such as salt water, liquid metal, molten solder, or a conductive ink to be annealed later. In some cases, the electrodes are micropatterned onto a planar surface and the microfluidic device is bonded to the surface. In some case, the electrodes are patterned on the substrate of the microfluidic device, e.g. a patterned indium tin oxide (ITO) glass slide. In some cases, the sorting element includes a selectively actuatable bipolar sorting electrode. In some cases, the sorting element includes two electrodes. In some cases, the sorting element includes a selectively actuatable bipolar droplet sorting electrode. In some cases, the electrode is a solid electrode prepared from any suitable conductive material may be utilized.

In some cases, the sorting element includes two sorting electrodes. In some cases, the two sorting electrodes have substantially different shapes, such as shown in FIG. 4. In some cases, the two sorting electrodes produce electric field lines with substantially different shapes. In some cases, the shapes are such that the pair of electrodes provide a constant electric field gradient. As such, a discrete entity can be subjected to the sorting force for a longer period of time and over a longer distance, thereby allowing a lower voltage to be used. In some cases, the electric field points radially inwards. In some cases, a portion of a first sorting electrode is positioned in the center of the arc of a concentric or essentially concentric sorting channel, and the second sorting electrode is positioned on a side of the sorting channel opposite the first sorting electrode, such as shown in FIG. 4. In some cases, the sorting channel defines a concentric or approximately concentric flow path, wherein a portion of a sorting electrode is located at the center of the concentric or approximately concentric flow path. In some cases, two sorting electrodes are positioned on the same side of the sorting channel. In such embodiments, the shortest distance between the two sorting electrodes is between about 20 μm and about 500 μm, such as between about 50 μm and about 200 μm, between about 75 μm and about 150 μm, between about 100 μm and about 150 μm, or between about 120 μm and about 140 μm. In some cases, the shortest distance between a sorting electrode and the interior of the sorting channel is between about 5 μm and about 100 μm, such as between about 10 μm and about 50 μm, between about 20 μm and about 40 μm, between about 25 μm and about 35 μm, or between about 28 μm and about 32 μm.

In some embodiments, the present disclosure provides microfluidic devices with an improved sorting architecture, which facilitates the high-speed sorting of discrete entities, e.g., microdroplets. This sorting architecture may be used in connection with other embodiments described herein or in any other suitable application where high-speed sorting of microdroplets is desired. Related methods and systems are also described. For example, in some embodiments, a microfluidic device may include a sorting channel; a first outlet channel in fluid communication with the sorting channel; a second outlet channel in fluid communication with the sorting channel; and a dividing wall separating the first outlet channel from the second outlet channel, wherein the dividing wall comprises a first proximal portion having a height which is less than the height of the inlet channel and a second distal portion having a height which is equal to or greater than the height of the inlet channel.

In some cases, the discrete entity is detected while the discrete entity is in the inlet channel via an optical property. In some cases, the optical property is fluorescence. Thus, in some cases, the detector includes an excitation light source and a fluorescence detector. In some cases, the excitation light includes visible light, ultraviolet light, or a combination thereof. In some cases, the detector is an optical scanner. In some cases, the detector includes optical fibers for directing excitation light onto the discrete entity, for directing fluorescent light to a fluorescence detector, or a combination thereof. In some cases, a suitable optical scanner utilizes a laser light.

A variety of different components can be included in the discrete entities to facilitate detection, including one or more fluorescent dyes. Such fluorescent dyes may be divided into families, such as fluorescein and its derivatives; rhodamine and its derivatives; cyanine and its derivatives; coumarin and its derivatives; Cascade Blue and its derivatives; Lucifer Yellow and its derivatives; BODIPY and its derivatives; and the like. Exemplary fluorophores include indocarbocyanine (C3), indodicarbocyanine (C5), Cy3, Cy3.5, Cy5, Cy5.5, Cy7, Texas Red, Pacific Blue, Oregon Green 488, Alexa fluor-355, Alexa Fluor 488, Alexa Fluor 532, Alexa Fluor 546, Alexa Fluor-555, Alexa Fluor 568, Alexa Fluor 594, Alexa Fluor 647, Alexa Fluor 660, Alexa Fluor 680, JOE, Lissamine, Rhodamine Green, BODIPY, fluorescein isothiocyanate (FITC), carboxy-fluorescein (FAM), phycoerythrin, rhodamine, dichlororhodamine (dRhodamine), carboxy tetramethylrhodamine (TAMRA), carboxy-X-rhodamine (ROX), LIZ, VIC, NED, PET, SYBR, PicoGreen, RiboGreen, and the like. Descriptions of fluorophores and their use, can be found in, among other places, R. Haugland, Handbook of Fluorescent Probes and Research Products, 9th ed. (2002), Molecular Probes, Eugene, Oreg.; M. Schena, Microarray Analysis (2003), John Wiley & Sons, Hoboken, N.J.; Synthetic Medicinal Chemistry 2003/2004 Catalog, Berry and Associates, Ann Arbor, Mich.; G. Hermanson, Bioconjugate Techniques, Academic Press (1996); and Glen Research 2002 Catalog, Sterling, Va.

In some embodiments, the microfluidic devices herein include directing the discrete entity to a discrete entity merger region. Accordingly, a device as described herein can include a discrete entity merger region and a trapping element positioned in proximity to the discrete entity merger region. The trapping element can trap a plurality of discrete entities in the discrete entity merger region for a time sufficient for the plurality of discrete entities to combine to form a combined discrete entity by exerting an electromagnetic force, exerting a mechanical force, applying heat, applying light, exerting an electrical force, providing a reagent, or a combination thereof sufficient. In some cases, the electromagnetic force is a dielectrophoretic force. In some cases, the electromagnetic force is an electrophoretic force. In some cases, the discrete entity merger region includes a feature selected from: a geometric change in a dimension of the first outlet channel, a flow obstacle, a flow divider, a laminating fluid inlet, a valve, or a combination thereof. In some cases, the geometric change is a change in the cross-sectional area of the first outlet channel (e.g., the discrete entity merger region has a larger cross-sectional area than the upstream region). In some cases, the geometric change is a change in one dimension of the first outlet channel (e.g., the discrete entity merger region is narrower than the downstream region). In some cases, the geometric change includes a recess in a channel wall. In some cases, the recess includes an area that is not colinear with the flow of fluid from the upstream region, such as shown as item 107 in FIG. 4. In some cases, where a valve is utilized, the valve is configured to switch between at least two states. In some cases, in the first state, the valve impedes the flow of a discrete entity past the discrete entity merger region while allowing flow of the carrier fluid past the discrete entity merger region. In some cases, in the second state, the valve is configured such that the combined discrete entity is not impeded from flowing past the discrete entity region. In some cases, the method includes putting the valve in a first state such that discrete entities can be trapped and combined into a combined discrete entity, and then putting the valve into a second state to release the discrete entity from the discrete entity merger region. In some cases, the valve is a membrane valve.

A laminating fluid inlet functions in a similar manner to certain embodiments of the spacer fluid inlet described above, such as a laminating fluid inlet is configured such that flowing fluid through the laminating fluid inlet will cause a discrete entity to move further away from a first side a channel and closer to a second side of a channel. Stated in another manner, the fluid flowing through the laminating fluid inlet contacts the fluid moving into the discrete entity merger region from an upstream region of the first outlet channel, thereby affecting the flow of fluid coming from the upstream region. In some cases, the fluid is oil, or a fluid which is otherwise immiscible with the fluid of the discrete entity.

FIG. 4 shows an embodiment wherein the discrete entity merger region includes recess 107, flow divider 113, and laminating fluid inlet 112. In FIG. 4, the laminating fluid provides a force pushing a discrete entity into recess 107 and towards trapping electrodes 109. In addition, flow divider 113 in FIG. 4 further affects the interaction of the laminating fluid and the fluid coming from the upstream region, thereby increasing the force pushing the discrete entity into recess 107. As such, a discrete entity merger region according to the present disclosure can include a laminating oil inlet and/or a flow divider, wherein such an element or elements are configured such that flowing oil through the laminating oil inlet channel will produce a force pushing a discrete entity in the discrete entity merger region towards a trapping electrode, a recess, or a combination thereof. In some embodiments, the device can include a flow divider without the laminating fluid inlet.

In some cases, the downstream region of the first outlet channel is configured to aid in the trapping of a discrete entity in the discrete entity merger region. In some cases, the downstream region has a larger cross-sectional area than the discrete entity merger region, which is an example of a geometric change in the first outlet channel. In some cases, the downstream region has a triangular or approximately triangular shape. In some cases, the downstream region has a triangular or approximately triangular shape and the discrete entity merger region is located at or near a vertex of the triangle. As an example, in the system of FIG. 5 has downstream region 208 and discrete entity merger region 207. In some cases, the longitudinal axis of the downstream region is parallel to the longitudinal axis of the discrete entity merger region, whereas in other cases such longitudinal axes are not parallel. In some cases, such axes are parallel but not colinear. In some cases, the axes are parallel and colinear. In some cases, the angle between such axes is greater than 0°, such as 5° or more, 10° or more, 15° or more, 30° or more, 45° or more, 60° or more, 75° or more, 90° or more, 135° or more, or 175° or more. In some cases, such an angle is between approximately 15° and approximately 135°. In some cases, such an angle is between approximately 60° and approximately 120°, such as shown in FIG. 5.

In some embodiments, the trapping element includes one or more electrodes, such as an electrode configured to exert a dielectrophoretic force on the discrete entity. In some cases, the electrode is configured to exert an electrophoretic force. The dielectrophoretic force on the discrete entity can be directed towards the electrode (i.e. an attractive force), away from the electrode (i.e. a repulsive force), or in any other direction. In some cases, the trapping electrode is a liquid electrode, such as a microfluidic channel containing a conductive material, e.g. salt water, liquid metal, molten solder, or a conductive ink to be annealed later. In some case, the electrodes are patterned on the substrate of the microfluidic device, e.g. a patterned indium tin oxide (ITO) glass slide. In some cases, the trapping element includes a selectively actuatable bipolar trapping electrode. In some cases, the trapping element includes two electrodes. In some cases, is the trapping element includes a selectively actuatable bipolar droplet trapping electrode. In some cases, the electrode is a solid electrode prepared from any suitable conductive material may be utilized. In some cases, the trapping element includes three or more trapping electrodes, such as four or more, five or more, ten or more, or twenty or more. In such cases, the trapping electrodes can be configured to form two or more bipolar electrode pairs, such as three or more pairs, four or more pairs, five or more pairs, or ten or more pairs.

In some cases, the sorting element sorts discrete entities at a rate of at least 10 Hz, such as at least 100 Hz, at least 500 Hz, at least 1,000 Hz, at least 2,000 Hz, or at least 10,000 Hz. In some cases, only 50% or less of the discrete entities contain the contents desired for the second discrete entity, such as 25% or less, 10% or less, 5% or less, 1% or less, or 0.1% or less. In some cases, the discrete entity merger region and trapping element are configured to trap a first discrete entity for 0.1 ms or more, such as 1 ms or more, 5 ms or more, 10 ms or more, 25 ms or more, 50 ms or more, 100 ms or more, 500 ms or more, 1,000 ms or more, or 5,000 ms or more. In some cases, a first discrete entity is trapped in the discrete entity merger region for 0.1 ms or more before a second discrete entity enters the region, such as 1 ms or more, 10 ms or more, 100 ms or more, or 1,000 ms or more.

The present disclosure provides a method of performing reactions by selectively combining two or more discrete entities, as described above, wherein the reaction occurs between one or more components from each discrete entity. Such components can be one or more cells, one or more products derived from a cell, one or more reagents, or a combination thereof. In some cases, a suitable method includes combination of one cell and one or more reagents. As an example, FIGS. 6A-H show the combination of four discrete entities, wherein three of the discrete entities each contain a different reagent and the fourth discrete entity contains a single cell. As such, FIGS. 6A-H show that a microfluidic device as described herein can be used to selectively combine different discrete entities, resulting in the formation of a combined discrete entity, e.g., that contains the three reagents and the cell. In some cases, the reagents can include cell lysing reagents, PCR reagents, reagents for analyzing the DNA or RNA within a cell, antibodies, or a combination thereof. In such cases, the method can further include collecting genomic data from contents of the discrete entities or combined discrete entities. In some cases, the one or more products derived from a cell include cell lysate, DNA, RNA, or a combination thereof. As such, the method can involve analyzing products from a cell, e.g. cell lysate, even though the cell per se is included in any of the discrete entities.

The present disclosure provides methods of selectively combining two or more discrete entities wherein each discrete entity contains one or more cell (e.g., a B-cell and a myeloma cell). In some cases, the ratio of a first type of cell to a second type of cell is 1.1:1.0 or more, e.g. 2:1 or more, 5:1 or more, 10:1 or more, 25:1 or more. The number of cells can be 2:1, 2:1 or more, 5:1 or more, 10:1 or more, 25:1 or more. In other cases, three or more types of cells are combined in unequal ratios or numbers. The ratio or number of each pair of cells can be those numbers and ratios recited above.

### REFERENCES

[1] Lu et al., “Development of therapeutic antibodies for the treatment of diseases,” J. Biomed. Sci., vol. 27, no. 1, p. 1, December 2020.
[2] Stevens, et al. “Characterization of a circulating subpopulation of spontaneous antitetanus toxoid antibody producing B cells following in vivo booster immunization.,” J. Immunol., vol. 122, no. 6, pp. 2498-504, June 1979.
[3] Yu et al., “An optimized electrofusion-based protocol for generating virus-specific human monoclonal antibodies,” J. Immunol. Methods, vol. 336, no. 2, pp. 142-151, July 2008.
[4] Skelley et al., “Microfluidic control of cell pairing and fusion,” Nat. Methods, vol. 6, no. 2, pp. 147-152, February 2009.
[5] Debs et al., “Functional single-cell hybridoma screening using droplet-based microfluidics,” Proc. Natl. Acad. Sci., vol. 109, no. 29, pp. 11570-11575, 2012.
[6] Siltanen et al., “An Oil-Free Picodrop Bioassay Platform for Synthetic Biology,” Sci. Rep., vol. 8, no. 1, pp. 1-7, 2018.
[7] Cole et al., “Printed droplet microfluidics for on demand dispensing of picoliter droplets and cells,” Proc. Natl. Acad. Sci., July 2017.
[8] Zhang et al., “Linked optical and gene expression profiling of single cells at high-throughput,” Genome Biol., vol. 21, no. 1, pp. 1-11, 2020.
[9] Zhu and Yang, “Hydrogel Droplet Microfluidics for High-Throughput Single Molecule/Cell Analysis,” Acc. Chem. Res., vol. 50, no. 1, pp. 22-31, January 2017.
[10] Weber et al., “Efficient targeting of fatty-acid modified oligonucleotides to live cell membranes through stepwise assembly.,” Biomacromolecules, vol. 15, no. 12, pp. 4621-6, December 2014.
[11] McGinnis et al., “MULTI-seq: sample multiplexing for single-cell RNA sequencing using lipid-tagged indices,” Nat. Methods, vol. 16, no. 7, pp. 619-626, 2019.
[12] Listek et al., “A novel selection strategy for antibody producing hybridoma cells based on a new transgenic fusion cell line,” Sci. Rep., vol. 10, no. 1, p. 1664, December 2020.
[13] Greenfield, “Electro Cell Fusion for Hybridoma Production,” Cold Spring Harb. Protoc., vol. 2019, no. 10, p. pdb.prot103184, October 2019.
[14] Yokoyama et al., “Production of monoclonal antibodies,” Curr. Protoc. Immunol., no. SUPPL. 102, pp. 1-29, 2013.
[15] Schoeman et al., “Electrofusion of single cells in picoliter droplets,” Sci. Rep., vol. 8, no. 1, p. 3714, December 2018.

All publications and patents mentioned in the present application are herein incorporated by reference. Various modification and variation of the described methods and compositions of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. A method comprising:
a) flowing a first and second discrete entity in a carrier fluid in a microfluidic device, wherein said microfluidic device comprises:
i) an inlet channel,
ii) a sorting channel in fluid communication with said inlet channel,
iii) first and second outlet channels in fluid communication with said sorting channel, wherein said first outlet channel comprises a merger region,
iv) a sorting element positioned in proximity to the sorting channel, and
v) a trapping element positioned in proximity to said merger region, and
wherein said first discrete entity comprises one, and only one, myeloma cell and is free of other types of cells, wherein said myeloma cell comprises: A) a first detectable label; and B) optionally, an outer surface displaying a first moiety;
wherein said second discrete entity comprises one, and only one, B-cell and is free of other types of cells, wherein said B-cell comprises: i) a second detectable label, and ii) optionally a second moiety that forms a binding pair with said first moiety; and
wherein said flowing causes said first and second discrete entities to pass through said inlet channel to said sorting channel;
b) selectively sorting said first and second discrete entities in said sorting channel to said first outlet channel;
c) trapping said first or second discrete entity in said merger region via said trapping element such that they combine to form at least part of a first combined discrete entity.

2. The method of claim 1, further comprising: combining, in any order, a third discrete entity with said first and second discrete entities at said merger region to form at least part of said first combined discrete entity, wherein said third discrete entity comprises gellable material and is free from cells.

3. The method of claim 2, further comprising releasing said first combined discrete entity from said merger region such that it flows into a downstream area.

4. The method of claim 3, wherein said downstream area is a collection area, or a receptacle external to said microfluidic device.

5. The method of claim 3, further comprising: d) treating said first combined discrete entity such that said gellable material gels, thereby forming a first gelled discrete entity.

6. The method of claim 5, wherein said treating comprises incubating said first combined discrete entity at a temperature that causes said gellable material to gel.

7. The method of claim 6, wherein said gellable material comprises low melt agarose.

8. The method of claim 2, further comprising: combining, in any order, a fourth discrete entity with said first, second, and third discrete entities at said merger region to form at least part of said first combined discrete entity, wherein said fourth discrete entity comprises a gel activator agent.

9. The method of claim 8, wherein said gel activator agent comprises an agent configured to release Ca²⁺ ions.

10. The method of claim 8, further comprising: releasing said first combined discrete entity from said merger region such that it flows into a downstream area.

11. The method of claim 10, further comprising: incubating said first combined discrete entity such that a first gelled discrete entity is formed.

12. The method of claim 8, wherein said gellable material comprises low melt alginate.

13. The method of claim 11, wherein said incubating is conducted for a time sufficient for said gel activator agent to convert all, or nearly all, of said gellable material into a gel.

14. The method of claim 1, wherein said first combined discrete entity further comprises gellable material.

15. The method of claim 14, further comprising releasing said first combined discrete entity from said merger region such that it flows into a downstream area.

16. The method of claim 15, further comprising: treating said first combined discrete entity such that said gellable material gels, thereby forming a first gelled discrete entity.

17. The method of claim 16, wherein said treating comprises incubating for a sufficient time and/or incubating at a particular temperature.

18. A system comprising:
a) first and second discrete entities in a carrier fluid, and/or
b) a combined discrete entity in a carrier fluid, wherein said combined discrete entity is a combination of said first and second discrete entities,
wherein said first discrete entity comprises one, and only one, myeloma cell and is free of other types of cells, wherein said myeloma cell comprises: A) a first detectable label; and B) optionally, an outer surface displaying a first moiety; and
wherein said second discrete entity comprises one, and only one, B-cell and is free of other types of cells, wherein said B-cell comprises: i) a second detectable label, and ii) optionally a second moiety that forms a binding pair with said first moiety.

19. The system of claim 18, further comprising: c) a microfluidic device comprising
i) an inlet channel,
ii) a sorting channel in fluid communication with said inlet channel,
iii) first and second outlet channels in fluid communication with said sorting channel, wherein said first outlet channel comprises a merger region,
iv) a sorting element positioned in proximity to the sorting channel, and
v) a trapping element positioned in proximity to said merger region.

20. A composition comprising:
a) first and second discrete entities in a carrier fluid, and/or
b) a combined discrete entity in a carrier fluid, wherein said combined discrete entity is a combination of said first and second discrete entities,
wherein said first discrete entity comprises one, and only one, myeloma cell and is free of other types of cells, wherein said myeloma cell comprises: A) a first detectable label; and B) optionally, an outer surface displaying a first moiety; and
wherein said second discrete entity comprises one, and only one, B-cell and is free of other types of cells, wherein said B-cell comprises: i) a second detectable label, and ii) optionally a second moiety that forms a binding pair with said first moiety.
